(19) United States
(12) Patent Application Publication
(10) Pub. No.: US 2023/0287438 A1
(43) Pub. Date: Sep. 14, 2023

(54) PLASMID VECTORS FOR IN VIVO SELECTION-FREE USE WITH THE PROBIOTIC E. COLI NISSLE

(71) Applicants: NORTHEASTERN UNIVERSITY, Boston, MA (US); PRESIDENT AND FELLOWS OF HARVARD COLLEGE, Cambridge, MA (US)

(72) Inventors: Neel S. Joshi, Somerville, MA (US); IIia Gelfat, Cambridge, MA (US); Anton KAN, Denton, TX (US)

(21) Appl. No.: 18/026,439

(22) PCT Filed: Sep. 15, 2021
(86) PCT No.: PCT/US2021/050479

§ 371 Date: Mar. 15, 2023

Related U.S. Application Data
(60) Provisional application No. 63/078,622, filed on Sep. 15, 2020.

(51) Int. Cl. C12N 15/70; C12N 15/62; A61K 9/00; C12N 1/20; A61K 35/74

(52) U.S. Cl. CPC C12N 15/70; C12N 15/62; A61K 9/0053; C12N 1/20; A61K 35/74

(57) ABSTRACT

Disclosed are methods for producing genetically modified bacteria, comprising introducing into said bacteria at least one engineered cryptic plasmid comprising a heterologous nucleic acid, wherein the heterologous nucleic acid comprises a nucleic acid sequence encoding a recombinant protein and a polypeptide secretion system for directing the recombinant protein to the outer membrane for secretion, wherein the bacteria do not comprise any native cryptic plasmids.

### RELATED APPLICATIONS

This application claims priority to U.S. Provisional Patent Application No. 63/078,622, filed on Sep. 15, 2020, the entire contents of which are expressly incorporated herein by reference.

### GOVERNMENT SUPPORT

This invention was made with government support under Grant Number R01DK110770 awarded by the National Institutes of Health. The government has certain rights in the invention.

### BACKGROUND

Escherichia coli Nissle 1917 (EcN) is a probiotic bacterium originally isolated from a particularly healthy soldier from World War I by the physician Alfred Nissle 1. Since then, this bacterium has found significant use as a probiotic therapy, outcompeting pathogens in the gut ² and thus protecting the host from infection. EcN has been at the forefront of probiotic genetic engineering ³, benefitting from the well-understood nature of E. coli biology, and from the many tools available to manipulate the organism. There are many projects working with engineered EcN ³, developing living therapeutics for diseases like hyperammonemia ⁴, and diagnostic tools for cancer detection ⁵.

In recent years, the gut microbiome has emerged as a critical factor for human health ⁶, however, the gut ecosystem remains a poorly understood system. One important approach to probe the gut microbiome is the development of engineered microbes that can sense and report on the conditions in the gut⁷, and deliver therapeutic molecules into the gut environment ⁸. Additionally, synthetic systems can provide insight into the behaviour of engineered bacteria in the gut environment ⁹, aiding further engineering efforts. As such, it is important to develop genetic tools to simplify bacterial engineering to study gut health and accelerate the development of sophisticated probiotic bacteria, capable of sensing and treating gut disorders.

Synthetic biology projects typically utilize plasmid vectors, circular DNA elements that can replicate within cells independently of the genome. Plasmids have many benefits: they are simple to manipulate, can be reliably transformed into E. coli cells, and can achieve high levels of gene expression due, in part, to a higher copy number than genomic DNA. Furthermore, several plasmids can be used in concert, allowing for modular assembly of complex synthetic genetic systems, as well as the simple independent testing of each plasmid in the system. An integral part of developing synthetic genetic systems is the iteration of prototypes in a design-test-build cycle ¹⁰, where during each cycle variants are tested to inform successive design iterations. Rapid and reliable genetic circuit construction and implementation is key for developing synthetic genetic systems, and plasmids offer an essential tool for this process. However, plasmid vectors also present a serious experimental limitation by requiring an antibiotic for selection and plasmid maintenance. In the context of in vivo therapeutic use in the gut, administration of an antibiotic is often incompatible with treatment and severely limiting to experiments as it induces drastic changes in the host microbiome¹¹.

Synthetic plasmids have been employed to engineer bacteria for in vivo use, however, without antibiotic selection, plasmid loss has been observed ¹², although this issue has not been extensively investigated. Strategies have been proposed to overcome this plasmid loss ¹², but these are still in early stages of development and come with their own drawbacks. Given the limitations of plasmids, EcN engineering projects that require stable transformants often insert DNA directly into the chromosome. However, genomic manipulations are typically limited by poor transformation efficiencies in EcN, and involve time-consuming and cumbersome protocols, impeding the iteration of genetic circuit designs. Furthermore, common genomic incorporation protocols such as Lambda Red based methods can be inefficient and have limitations on insert length ^{13,14} further slowing or outright preventing the development of large multi-component synthetic genetic systems. Additionally, genomic incorporation limits recombinant DNA copy number to genomic copy number, making the achievement of high gene expression rates more difficult. Given the importance of rapid prototyping for the development of synthetic genetic systems, new paradigms are required to host synthetic DNA to facilitate the engineering of probiotic organisms.

### SUMMARY

The invention comprises an engineered strain of non-pathogenic E. coli harboring two pieces of modified plasmid DNA that enable it to secrete proteins inside the mammalian gastrointestinal (GI) tract. Diseases of the GI tract (e.g. Crohn's, ulcerative colitis) are hard to treat because it is difficult to maintain a steady amount of drug at the site of disease, due to the constant flow of material through the gut. One solution to this problem is to use a living bacterium as the drug delivery vehicle. The bacterium can multiply itself inside the gut, maintaining a relatively steady concentration over time, all while secreting a therapeutic directly at the site of disease. In order to accomplish this, one needs to genetically modify the bacterium so that it behaves appropriately. Genetic engineering of this type in E. coli Nissle, the most common starting point because of its safety profile and genetic tractability, can either occur through insertions to the chromosome or with smaller pieces of circular DNA called plasmids. Chromosomal modification is time consuming and has other limitations. Plasmids are much easier to work with and modify, but they typically require constant antibiotic selection in order to stay associated with the bacterial host—something that is undesirable for use in a human patient. The plasmids were engineered to remain stably associated with E. coli Nissle in the absence of antibiotic selection, facilitating their use in vivo. Overall, the technology is a tool that one could use to engineer bacteria to perform better as a living therapeutic, such as a gut therapeutic.

In some aspects of the invention, disclosed herein are methods for producing a genetically modified bacterium, comprising introducing into a bacterium at least one engineered cryptic plasmid comprising a heterologous nucleic acid, wherein the heterologous nucleic acid comprises a nucleic acid sequence encoding a recombinant protein and a polypeptide secretion system for directing the recombinant protein to the outer membrane for secretion, wherein the bacterium does not comprise any native cryptic plasmids.

In certain aspects of the invention, provided herein are engineered bacterium, comprising at least one engineered cryptic plasmid comprising a heterologous nucleic acid, wherein the heterologous nucleic acid comprises a nucleic acid sequence encoding a recombinant protein and a polypeptide secretion system for directing the recombinant protein to the outer membrane for secretion, wherein the bacterium does not comprise any native cryptic plasmids.

### BRIEF DESCRIPTION OF THE DRAWINGS

FIG. 1 depicts plasmid maps of the native cryptic A) pMUT1 and B) pMUT2 plasmids in EcN with known genes labelled, also shown are the insertion sites where recombinant cassettes were inserted, with unsuccessful sites greyed out. C) Insulated characterization cassettes inserted onto the pMUT plasmids to produce engineered pMUT vectors, with cassette ‘AsG’ containing an ampicillin resistance gene and constitutively expressed GFP, and ‘TsR’ containing tetracycline resistance and constitutive RFP. D) For site characterization, the ‘AsG’ cassette was inserted into the 2 sites on each pMUT plasmid, and characterized both the E) bacterial growth rate and F) GFP expression rates. In each case, the performance of the engineered plasmids was characterized in both an unmodified EcN strain, and an EcN strain where the relevant native pMUT plasmid had been removed.

FIG. 2 illustrates the curing native pMUT plasmids. EcN pMUT plasmids were assessed with primers around the insertion sites and primers A) muta5 and muta6 on pMUT1 and B) muta7 and muta8 on pMUT2. C) Plasmid pFREE cleaves pMUT1 through expression of Cas9 and gRNA targeting the colE1 origin of replication on pMUT1 and on pFREE itself. D) Similarly, pCryptDe14.8 targets the origin of pMUT2 and itself, and also contains a RelB antitoxin to disrupt the RelE-RelB toxin-antitoxin system on pMUT2. E-F) Agarose gels showing the results of colony PCRs around the insertion sites of the ‘AsG’ cassette, revealing that transformation with an engineered plasmid does not displace the native plasmid. I-J) pFREE and pCryptDe14.8 can cure EcN of native plasmids, and these can be replaced with engineered versions.

FIG. 3 depicts the temperature-sensitive gene expression system. A) Temperature sensitive expression was achieved with the TlpA36 protein, which dimerizes, binds, and represses the pTlpA promoter at temperatures below 36° C. B) To make a library with various promoter strengths, the pTlpA promoter was modified to contain 3 variable nucleotides near the −10 region of the promoter. C-D) Variable GFP expression strengths at 37° C. from the pTlpA library, with green curves showing 40 pM1s3AsR_TS* variants, and blue showing 40 pM2s2AsR_TS* variants, with little expression at 30° C. E-F) In contrast, RFP expression was not as variable. When promoter strengths were quantified at 37° C. for the g) pMUT1 and I) pMUT2 engineered vectors, a range of strengths was found, and 9 promoters throughout the range were chosen for sequencing and further development. H-J) In general, gene expression from the pTlpA* promoters was reduced at 30° C. by around an order of magnitude.

FIG. 4 depicts the characterization data from the E. coli Mach1 cloning strain. A) The temperature sensitive gene expression circuit in EcN compared to the E. coli Mach1 strain. B) GFP expression from the pTlpA library in EcN, showing a range of expression strengths. C) Constitutive RFP from the ‘AsR_TS*’ cassettes from the engineered pMUT plasmids in EcN.

FIG. 5 depicts the characterization of temperature sensitive curli production. A) Diagram of the temperature sensitive curli production construct, containing a pTlpA promoter variant driving the expression of a synthetic curli operon csgBACEFG, with the csgA protein sequence altered to either contain an E-tag epitope tag (labelled cassette ‘csg-Etag’), or an E-tag and a GFP nanobody sequence and a 6xHis sequence (cassette ‘csg-Etag-NbGFP’). The temperature sensitive promoter variants were all used to generate pM1s3ATScsg- #variants (B and D) and pM2s2ATScsg- #(C and E) variants, which were assayed with a CR assay (b and c) and an anti-Etag filtration ELISA (D and E). F) Representative micrographs of bacterial cultures harbouring plasmids with temperature inducible curli grown at 37° C. in the presence of Congo Red, which stains the amyloid with and becomes fluorescent (in red). G) Curli fused to GFP nanobodies (NbGFP) was able to remove a significant amount of sfGFP from a 4 μg/mL solution of purified sfGFP in PBS.

FIG. 6 depicts the engineered pMUTs in the mouse gut. A) Timeline of in vivo study. ABx—antibiotics (carbenicillin). B) Bacterial density of PBP8 over time, as measured by CFU counts from fecal samples plated on LB agar with Cm. C) Plasmid retention over time. (D-F) Relative in vivo protein expression levels from fecal filtration ELISA on days 0, 1 and 2 (D, E and F, respectively). At each day, engineered pMUT conditions were tested against a WT pMUT control by one-way ANOVA, followed by pairwise Welch's t-test. ns—not significant, * p<0.05, ** p<0.01, **** p<0.0001. All data are represented as mean±SEM.

FIG. 7 depicts plasmid maps of pMUT1 and pMUT2 (left), alongside a table of annotations (right) for predicted ORFs in the native pMUT plasmids, with the ‘Predicted ORF’ referring to the labels on the plasmid maps

FIG. 8 illustrates expression characterization from cassettes A) ‘AsG’ and B) ‘TsR’ on plasmids pM1s3 and pM2s2. In all cases, EcN was used without the relevant native pMUT plasmid. In both cases, pM1s3 plasmid backbone provides higher gene expression. The relative difference in recombinant protein expression strength between the engineered pMUT1 and pMUT2 plasmids is independent of the fluorescent protein used for characterization. Error bars show standard deviation from 8 replicates.

FIG. 9 depicts plasmid vectors to cure EcN cryptic plasmids. A) Plasmid map of pFREE, showing ATC inducible Cas9 and rhamnose inducible CRISPR gRNA array, B) plasmid map of the pCryptDel plasmid variants, which are based on pFREE with a modified CRISPR array and a relB anti-toxin gene. C) A detailed look at the gRNA arrays. gRNA-X and gRNA-Y refer to variants (shown in Table 6), where X and Y pair were either 5 and 6, 7 and 8, or 9 and 10. The final construct, pCryptDe14.8, contained gRNA9 and 10, but had a 34 bp insertion in the region just upstream of gRNA3.

FIG. 10 depicts Representitative TAE agarose gels of colony PCR results following a typical pMUT curing process, removing native plasmids from EcN with A) pFREE, B) pCryptDe14.8, and C) from EcN ΔpMUT1 with pCryptDe14.8. In all cases primers muta5, muta6, muta7 and muta8 were used, which result in a 429 bp band in the presence of pMUT2, and a 361 bp in the presence of pMUT1. In each case, an orange star shows a colony cured of pMUT1, and a blue star shows a colony cured of pMUT2. Panel D shows a gel for 3 replicate colonies each of EcN, EcNΔpMUT1, EcNΔpMUT2, and EcN ΔpMUT1 ΔpMUT2.

FIG. 11 depicts A) Synthetic plasmids pKAG, which constitutively expresses sfGFP, and pL6FO, which expresses the synthetic curli operon csgBACEFG with IPTG induction, were transformed into E. coli Nissle strain PBP8. B) Administration and sampling schedule, with mice in all groups were treated with chloramphenicol to select for PBP8 cells from day −3 to the end of the experiment. On day 0, mice were administered with 1010 CFU of PBP8 transformed with either pKAG (n=3), or pL6FO (n=4), and half of the PBP8+pL6FO mice were given the IPTG inducer in their water. Fecal samples were collected regularly to detect PBP8 (chloramphenicol resistant) or PBP8 with plasmid (chloramphenicol and kanamycin resistant) by plating assays. C) After administration, PBP8 cells were maintained in the mice for all conditions throughout the experiment, but D) all plasmids suffered significant plasmid loss, particularly after day 2. Shaded areas show relative standard error for panel C and standard deviation for panel D.

### DETAILED DESCRIPTION

Bacteria isolated from clinical samples often contain plasmids, including small cryptic plasmids that are maintained at high copy number despite containing little genetic information and conferring no apparent phenotype ¹⁵. Many of these plasmids have no known function, although one study linked the presence of such small cryptic plasmids to phage resistance ¹⁶. EcN contains two such cryptic plasmids, pMUT1 and pMUT2, which are stable within the bacteria and survive passage through the gut, and are used as targets to detect the EcN in clinical PCR assays ¹⁷. The pMUT plasmids do not confer any detectable phenotype, are not essential to EcN and do little to affect growth ¹⁸. Furthermore, the pMUT plasmids do not present a metabolic burden to EcN, at least under laboratory conditions ¹⁹. Whilst several projects have used pMUT plasmids to carry synthetic circuits ³, no systematic engineering attempt has been made to domesticate and characterize the efficacy of engineered pMUT plasmids in vivo.

Disclosed herein is the systematic engineering of the E. coli Nissle 1917 cryptic plasmids pMUT1 and pMUT2 to create a series of plasmid vectors for use in the gut. Several sites were tested on each plasmid to insert recombinant DNA cassettes containing selection and fluorescent markers, and characterized the gene expression in each case. It was found that the native plasmids were not lost through transformation of an engineered variant, thus a technique was developed to remove the native plasmids through a CRISPR-Cas9 mechanism. Further functionality was added to these plasmid vectors: adapting and expanding a temperature sensitive expression system, as well as curli-based protein secretion to export proteins into the extracellular space. The plasmids were then tested in vivo and demonstrated that EcN retained the engineered pMUT plasmids during passage through the mouse GI tract, and that the plasmids were capable of secreting recombinant protein into the extracellular space of the gut.

### Advantages Include:

The invention disclosed herein, for the first time:
1. enables protein secretion from non-pathogenic bacteria inside the mammalian gut;
2. foes not require antibiotic selection; and
3. uses a plasmid type (pMUT) that have not been previously modified in this way, and are natively derived from E. coli Nissle.

This Invention Also Provides:
1. faster design-build-test iteration for engineered living therapeutics;
2. potentially appropriate use in humans;
3. use of cellular secretion machinery that has not been used before for this purpose, e.g., high secretion efficiency enables high local concentrations of therapeutic molecules at the site of disease; and
4. A potentially better safety profile due to local delivery, e.g., no immunosuppression, risk of infection/lymphoma, as is the case for some biologics.

### Potential Uses Include:

The invention as contemplated herein may be useful for:
1. secretion of soluble biologic drugs in the gut;
2. self-regenerating mucosal wound healing patchers to treat inflammatory lesions; and
3. use either alone or in combination with other inflammatory bowel disease (IBD) therapies to maintain remission for longer periods of time or delay the use of biologics for treatment.

In some aspects, the invention provided herein may be used as a tool for rapid design/testing of living therapeutics.

### Cost Advantages Include:

The disclosed invention is much more cost efficient than biologics. For example, and without limitation, manufacturing involves much less downstream processing, and the manufactured product may be taken orally. Accordingly, the invention disclosed herein is sufficiently cost-effective to be of particular use in treating diseases in developing countries (e.g., enteric pathogens).

### Performance Advantages Include:

There is no living therapeutic currently on the market capable of protein secretion in the gut. The invention provided herein enables the production and therapeutic use against a range of indications.

### Definitions

As used herein, the term “engineered bacterium” or “engineered bacterial cell” refers to a bacterial cell that has been genetically modified from its native state. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells of the disclosure may comprise exogenous nucleotide sequences on plasmids. Alternatively, recombinant bacterial cells may comprise exogenous nucleotide sequences stably incorporated into their chromosome. In some embodiments, the engineered bacterium is non-pathogenic. In some embodiments, the engineered bacterium is pathogenic.

“Probiotic”, as used herein, refers to a live, non-pathogenic microorganism, e.g., a bacterium, which can confer health benefits to a host organism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Bacteroides (e.g., Bacteroides fragilis, Bacteroides subtilis, and Bacteroides thetaiotaomicron) and Escherichia coli. In some embodiments, the probiotic is Gram-negative bacterium. The probiotic may be a variant or a mutant strain of bacterium. Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

In some embodiments, the antibody is a donkey antibody. In some embodiments, the antibody is a rat antibody. In some embodiments, the antibody is a horse antibody. In some embodiments, the antibody is a camel antibody. In some embodiments, the antibody is a shark antibody.

The term “antigen-binding portion” of an antibody (or simply “antibody fragment”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of antibody fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341: 544-546; and PCT Publication No. WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Nat'l. Acad. Sci. USA 85:5879-5883). Such single chain antibodies are also intended to be encompassed within the term “antigen-binding portion” of an antibody. Other forms of single chain antibodies, such as diabodies are also encompassed. Antibody fragments also include single domain antibodies, maxibodies, minibodies, nanobodies, intrabodies, diabodies, triabodies, tetrabodies, v-NAR and bis-scFv (see, e.g., Hollinger and Hudson (2005) Nature Biotechnology 23:1126-1136).

A “single domain antibody”, as used herein, refers to the heavy chain variable domain (“VH”) of an antibody, i.e., a heavy chain variable domain without a light chain variable domain. Single domain antibodies are described, for example, in Hamers-Casterman et al. (1993) Nature 363:446-48, and Dumoulin et al. (2002) Protein Science 11:500-15. Single domain antibodies can be derived from a multiple animals, including, for example, llama, alpaca, camel (i.e., camelid single domain antibodies), and shark.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence.

As used herein, a “heterologous” gene, “heterologous sequence”, or “heterologous nucleic acid” refers to a nucleic acid sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. A heterologous gene may include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature.

As used herein, the term “endogenous gene” refers to a native gene in its natural location in the genome of an organism.

As used herein, the term “transgene” refers to a gene that has been introduced into the host organism, e.g., host bacterial cell's genome.

As used herein, a “SecA-dependent secretion signal”, refers to a polypeptide sequence which, when present on a polypeptide, e.g., at the N-terminus of a polypeptide, can cause the polypeptide to be exported from the cytoplasm of a bacterium across the inner membrane as mediated by a bacterial SEC system. In some embodiments, the SecA-dependent secretion signal is the polypeptide having the sequence of the E. coli CsgA SecA-dependent secretion signal and homologs and/or variants, including conservative substitution variants, thereof.

As used herein, a “signal recognition particle (SRP) pathway signal sequence” refers to a polypeptide sequence which, when present on a polypeptide (e.g., the N-terminus of a polypeptide), can cause the polypeptide to be exported from the cytoplasm of a bacterial cell across the inner membrane as mediated by the single recognition particle (SRP) pathway proteins. In some embodiments, the polypeptide is translated and transported across the inner membrane concurrently, thus guiding the nascent polypeptide into the periplasm. In some embodiments, the SRP pathway signal sequence is the SRP signal sequence from CcmH, DsbA, FocC, NikA, SfmC, TolB, TorT, YraI, or homologs and/or variants, including conservative substitution variants, thereof.

As used herein, a “CsgGE export signal sequence” refers to a polypeptide sequence which, when present at the N-terminus of a polypeptide can cause the polypeptide to be targeted by CsgE and exported across the outer membrane of the cell via the CsgG oligomeric transport complex of a curli export system, or by an orthologous export system. In some embodiments, the CsgG targeting sequence is the last 22 amino acids of the bipartite curli signal sequence of an endogenous polypeptide exported by the curli export system. In some embodiments, the CsgG targeting sequence can be a polypeptide having the sequence of an E. coli CsgA CsgGE export signal sequence and homologs and/or variants, including conservative substitution variants, thereof.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell-specific or tissue-specific manner, in response to different environmental or physiological conditions, or in response to specific compounds. Prokaryotic promoters are typically classified into two classes: inducible and constitutive.

“Constitutive promoter” refers to a promoter that is capable of facilitating continuous transcription of a coding sequence or gene under its control and/or to which it is operably linked. Constitutive promoters and variants are well known in the art and include, but are not limited to, a constitutive Escherichia coli σ^{s} promoter, a constitutive Escherichia coli σ³² promoter, a constitutive Escherichia coli σ⁷⁰ promoter, a constitutive Bacillus subtilis σ^{A} promoter, a constitutive Bacillus subtilis σ^{B} promoter, and a bacteriophage T7 promoter.

An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” For example, and without limitation, chemical agents, temperature, and light may be used for induction of the promoters contemplated herein. Preferably, the promoter is a temperature sensitive promoter.

As used herein, the term “expression” refers to the transcription and stable accumulation of sense (mRNA) or anti-sense RNA derived from a nucleic acid, and/or to translation of an mRNA into a polypeptide

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one amino acid catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

As used herein, the term “exogenous” refers to a substance (e.g., a nucleic acid or polypeptide) present in a cell other than its native source. The term exogenous can refer to a nucleic acid or a protein that has been introduced by a process involving the hand of man into a biological system such as a cell or organism in which it is not normally found or in which it is found in undetectable amounts. A substance can be considered exogenous if it is introduced into a cell or an ancestor of the cell that inherits the substance. In contrast, the term “endogenous” refers to a substance that is native to the biological system or cell.

A “non-amyloid polypeptide”, as used herein, refers to a polypeptide that does not form amyloid aggregates in a cell (e.g., a bacterial cell). An “amyloidogenic polypeptide” refers to a peptide that either forms or increases the formation of amyloid aggregates in a cell. In some embodiments, the therapeutic polypeptide is a non-amyloid polypeptide. In some embodiments, the polypeptide is a non-amyloiodogenic polypeptide. An “amyloid polypeptide” refers to a polypeptide that forms amyloid aggregates in a bacterial cell. An “amyloidogenic polypeptide” refers to a polypeptide that either forms or increases the formation of amyloid aggregates in a cell. In some embodiments, the therapeutic polypeptide is an amyloid polypeptide. In some embodiments, the therapeutic polypeptide is an amyloidogenic polypeptide.

A “pharmaceutical composition,” as used herein, refers to a composition comprising an active ingredient (e.g., a bacterial cell, an inducer, a drug, or a detectable compound) with other components such as a physiologically suitable carrier and/or excipient.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

A “plasmid” or “vector” includes a nucleic acid construct designed for delivery to a host cell or transfer between different host cell. An “expression plasmid” or “expression vector” can be a plasmid that has the ability to incorporate and express heterologous nucleic acid fragments in a cell. An expression plasmid may comprise additional elements, for example, the expression vector may have two replication systems, thus allowing it to be maintained in two organisms. The nucleic acid incorporated into the plasmid can be operatively linked to an expression control sequence when the expression control sequence controls and regulates the transcription and translation of that polynucleotide sequence. In some embodiments of the invention disclosed herein, the plasmid or vector is derived from a cryptic plasmid, such as, but not limited to, pMUT1 and/or pMUT2.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. The terms “protein” and “polypeptide” as used herein refer to both large polypeptides and small peptides. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “therapeutic polypeptide” refers to any polypeptide that has a therapeutic effect or may be used for diagnostic purposes when introduced into a eukaryotic organism (e.g., a mammalian subject such as human). In some embodiments, the therapeutic polypeptide is an antibody. In some embodiments, the therapeutic polypeptide is a single domain antibody. In some embodiments, the therapeutic polypeptide is a fusion protein, a hormone, an antigen, a thrombolytic agent, a cytokine or a growth factor. In some embodiments, the therapeutic polypeptide is an immunotoxin (e.g., an antibody fused to a cellular toxin).

The term “operatively linked” includes having an appropriate transcription start signal (e.g., promoter) in front of the polynucleotide sequence to be expressed, and having an appropriate translation start signal (e.g., a Shine Delgarno sequence and a start codon (ATG)) in front of the polypeptide coding sequence and maintaining the correct reading frame to permit expression of the polynucleotide sequence under the control of the expression control sequence, and, optionally, production of the desired polypeptide encoded by the polynucleotide sequence. In some examples, transcription of a gene encoding a recombinant polypeptide as described herein is under the control of a promoter sequence (or other transcriptional regulatory sequence) which controls the expression of the nucleic acid in a cell-type in which expression is intended. It will also be understood that the gene encoding a recombinant polypeptide as described herein can be under the control of transcriptional regulatory sequences which are the same or which are different from those sequences which control transcription of the naturally-occurring form of a protein.

The terms “overexpression” or “overexpress”, as used herein refers to the expression of a functional nucleic acid, polypeptide or protein encoded by DNA in a host cell, wherein the nucleic acid, polypeptide or protein is either not normally present in the host cell, or wherein the nucleic acid, polypeptide or protein is present in the host cell at a higher level than that normally expressed from the endogenous gene encoding the nucleic acid, polypeptide or protein.

A “nucleic acid” or “nucleic acid sequence” may be any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e. a non-detectable level as compared to a reference level. In the context of a marker or symptom, a “decrease” is a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The term “non-pathogenic” as used herein to refer to bacteria refers to bacteria that are not capable of causing disease or harmful responses in a host. In some embodiments, non-pathogenic bacteria are commensal bacteria. Examples of non-pathogenic bacteria include, but are not limited to Bacteroides and Escherichia coli, e.g., Escherichia coli Nissle 1917, Bacteroides fragilis, Bacteroides subtilis, and Bacteroides thetaiotaomicron, Naturally pathogenic bacteria may be genetically engineered to provide reduce or eliminate pathogenicity.

As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, polypeptides described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g. ability to target a polypeptide for export via the curli export system. A wide variety of PCR-based site-specific mutagenesis approaches are also known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

In some aspects of the invention, disclosed herein are methods for producing a genetically modified bacterium, comprising introducing into a bacterium at least one engineered cryptic plasmid comprising a heterologous nucleic acid, wherein the heterologous nucleic acid comprises a nucleic acid sequence encoding a recombinant protein and a polypeptide secretion system for directing the recombinant protein to the outer membrane for secretion, wherein the bacterium does not comprise any native cryptic plasmids.

In some embodiments, the at least one engineered cryptic plasmid is an engineered pMUT1 or pMUT2. In some such embodiments, the nucleic acid sequence encoding the recombinant protein and polypeptide secretion system (e.g., the expression cassette) is inserted into the pMUT1 backbone or pMUT2 backbone at insertion sites as indicated in Table 3. In some embodiments, the nucleic acid sequence encoding the recombinant protein and polypeptide secretion system (e.g., the expression cassette) is inserted within a site amplified by a primer pair comprising the sequences set forth in SEQ ID NOs: 21 and 22; SEQ ID NOs: 23 and 24; SEQ ID NOs: 25 and 26; or SEQ ID NOs: 27 and 28. In some embodiments, the nucleic acid sequence encoding the recombinant protein and polypeptide secretion system comprises a curli fiber secretion system. For example, and without limitation, the nucleic acid sequence encoding the recombinant protein and polypeptide secretion system comprises a synthetic csgBACEFG operon.

In some preferred embodiments, the heterologous nucleic acid sequence encodes a recombinant protein fused to a CsgA monomer, e.g., an engineered CsgA as contemplated herein. In some embodiments, the recombinant protein comprises a therapeutic polypeptide selected from the group consisting of an antibody, an antibody fragment, an enzyme, a fusion protein, a hormone, an antigen, a thrombolytic agent, a cytokine, an immunotoxin, and a growth factor. In some such embodiments, the therapeutic polypeptide is an antibody fragment; and the antibody fragment is a single chain antibody, such as a nanobody. In some embodiments, the single chain antibody is specific for an antigen selected from the group consisting of: carcinogenic embryonic antigen (CEA), glucose transporter 1 (GLUT1), green fluorescent protein (GFP), beta-lactamase, Clostridium difficile Toxin A, Clostridium difficile Toxin B, botulinum toxin (BoTox), cholera toxin (CTX), norovirus capsid protein, rotavirus capsid protein, and Plasmodium membrane protein. Contemplated embodiments also include the therapeutic polypeptide fused to an amyloid polypeptide. As a non-limiting example, the amyloid polypeptide may comprise at least one curli subunit.

In some embodiments, the engineered cryptic plasmid lacks a selectable marker gene. In some embodiments, the heterologous nucleic acid is operably linked to an inducible promoter. Such inducible promoters may be responsive to an inducer selected from the group consisting of IPTG, arabinose, tetracycline, and permissive temperature change. In preferred embodiments, the inducible promoter is a temperature sensitive promoter.

In some embodiments, the bacterium of the invention retain the engineered cryptic plasmid in the absence of a selectable marker. In some embodiments, the methods provided herein further comprise plasmid-curing the bacterium prior to introduction of the engineered cryptic plasmid.

In some aspects, described herein are engineered microbial cells comprising an engineered CsgA polypeptide and/or comprising a vector (e.g., an engineered cryptic vector) or nucleic acid encoding such a polypeptide. In certain aspects of the invention, provided herein are engineered bacterium, comprising at least one engineered cryptic plasmid comprising a heterologous nucleic acid, wherein the heterologous nucleic acid comprises a nucleic acid sequence encoding a recombinant protein and a polypeptide secretion system for directing the recombinant protein to the outer membrane for secretion, wherein the bacterium does not comprise any native cryptic plasmids. In some embodiments, the at least one engineered cryptic plasmid is an engineered pMUT1 or pMUT2.

In some embodiments, the nucleic acid sequence encoding the recombinant protein and polypeptide secretion system (e.g., an expression cassette) is inserted into the pMUT1 backbone or pMUT2 backbone at insertion sites as indicated in Table 3. In some embodiments, the nucleic acid sequence encoding the recombinant protein and polypeptide secretion system (e.g., the expression cassette) is inserted within a site amplified by a primer pair comprising the sequences set forth in SEQ ID NOs: 21 and 22; SEQ ID NOs: 23 and 24; SEQ ID NOs: 25 and 26; or SEQ ID NOs: 27 and 28. In some such embodiments, the nucleic acid sequence encoding the recombinant protein and polypeptide secretion system comprises a curli fiber secretion system. In some preferred embodiments, the nucleic acid sequence encoding the recombinant protein and polypeptide secretion system comprises a synthetic csgBACEFG operon. In some embodiments, the heterologous nucleic acid sequence encodes a recombinant protein fused to a CsgA monomer, e.g., an engineered CsgA.

In some embodiments of the engineered bacterium provided herein, the recombinant protein comprises a therapeutic polypeptide selected from the group consisting of an antibody, an antibody fragment, an enzyme, a fusion protein, a hormone, an antigen, a thrombolytic agent, a cytokine, an immunotoxin, and a growth factor. In some such embodiments, the therapeutic polypeptide is an antibody fragment; and the antibody fragment is a single chain antibody, such as a nanobody. The single chain antibodies provided herein may be specific for an antigen selected from the group consisting of: carcinogenic embryonic antigen (CEA), glucose transporter 1 (GLUT1), green fluorescent protein (GFP), beta-lactamase, Clostridium difficile Toxin A, Clostridium difficile Toxin B, botulinum toxin (BoTox), cholera toxin (CTX), norovirus capsid protein, rotavirus capsid protein, and Plasmodium membrane protein.

In some embodiments, the therapeutic polypeptide is fused to an amyloid polypeptide. In some such embodiments, the amyloid polypeptide comprises at least one curli subunit. Accordingly, in some preferred embodiments the therapeutic polypeptide may be fused to a CsgA subunit, e.g., an engineered CsgA.

In some embodiments, the engineered bacterium and/or the engineered cryptic plasmid lacks a selectable marker gene.

In some embodiments, the heterologous nucleic acid, e.g., expression cassette, is operably linked to an inducible promoter. In some such embodiments, the inducible promoter is responsive to an inducer selected from the group consisting of IPTG, arabinose, tetracycline, and permissive temperature change. Preferably, the inducible promoter is a temperature sensitive promoter.

In some embodiments of the invention, the bacterium retains the engineered cryptic plasmid in the absence of a selectable marker.

In certain embodiments, the heterologous nucleic acid further comprises a nucleic acid sequence encoding a polypeptide tag. Such polypeptide tags may be selected from the group consisting of a poly-histidine tag, a myc tag a FLAG tag, a hemagglutinin (HA) tag, and a V5 tag.

In some embodiments, the engineered bacterium is a non-pathogenic bacterium. In some embodiments, the engineered bacterium is a bacterium of the genus Bacteroides or Escherichia. The engineered bacterium may be a probiotic bacterium. In some preferred embodiments, the engineered bacterium is Escherichia coli, such as Escherichia cob strain Nissle 1917. In some such embodiments, the engineered bacterium does not comprise a native csgBACEFG operon.

In some embodiments, the engineered CsgA polypeptide can comprise a functional polypeptide. In some embodiments, the engineered. CsgA polypeptide can comprise a functional polypeptide comprising a conjugation domain. In some embodiments, a cell encoding and/or comprising an engineered CsgA polypeptide can comprise an activity polypeptide. In some embodiments, a cell encoding and/or comprising an engineered CsgA polypeptide can comprise an activity polypeptide comprising a conjugation domain can further encode and/or comprise a second engineered polypeptide comprising a partner conjugation domain and a functionalizing polypeptide. In some embodiments, described herein is a population of cells comprising two cell types, the first cell type encoding and/or comprising an engineered CsgA polypeptide comprising an activity polypeptide comprising a conjugation domain and the second cell type encoding and/or comprising a second engineered polypeptide comprising a partner conjugation domain and a functionalizing polypeptide. That is, a single cell can comprise a CsgA polypeptide with a conjugation domain and also comprise the polypeptide which will bind to and/or be bound by that CsgA polypeptide or that a first cell can comprise a CsgA polypeptide with a conjugation domain and a second cell can comprise the polypeptide which will bind to and/or be bound by that CsgA polypeptide. It is further contemplated that an engineered CsgA polypeptide with a conjugation domain can be contacted with a second polypeptide comprising a partner conjugation domain and a functionalizing polypeptide, e.g. the second polypeptide can be produced (e.g. by a bacteria or eukaryotic cell) and/or synthesized (and optionally isolated or purified) and then brought in contact with the engineered CsgA polypeptide, e.g. when the CsgA polypeptide is present on a cell surface and/or present in a biofilm.

Functional polypeptides within the scope of the present disclosure include peptides or proteins having a desired function. Such functions include catalytic function, recognition function or structural function. Exemplary functional polypeptides include targeting domains. Exemplary functional polypeptides include therapeutic polypeptides. Exemplary functional polypeptides include diagnostic polypeptides. Exemplary functional polypeptides include anticancer polypeptides. Exemplary functional polypeptides include antimicrobial polypeptides. Exemplary functional polypeptides include anti-inflammatory polypeptides. Exemplary functional polypeptides include polymer binding polypeptides, Exemplary functional polypeptides include metabolite binding polypeptides. Exemplary functional polypeptides include targeting polypeptides. Exemplary functional polypeptides include functional polypeptides that bind to tissues or cells or substrates. Exemplary functional polypeptides include a first member of a known binding pair. When expressed, the first member of the binding pair is available for binding to a second member of the binding pair which may have attached to it a functional polypeptide, such as for therapeutic or diagnostic purposes. In this manner, the functional polypeptide with the second member of the binding pair may be contacted to the biofilm to add the functional polypeptide to the biofilm, such as to provide the biofilm with the characteristic of the functional polypeptide. Exemplary functional polypeptides may be those to which a functional group may be covalently attached either directly or through a linker. For example, by appending to CsgA a peptide capable of undergoing spontaneous covalent modification, a biofilm whose surface can be modified with any protein or compound of interest can be created by subsequent addition of the protein or compound of interest.

Exemplary therapeutic polypeptides include engineered polypeptides with therapeutic function, polypeptides with anti-inflammatory bioactivity (trefoil factors—e.g. TFF1-3, interleukins—e.g. IL-10, other anti-inflammatory cytokines, anti-TNFα factors), polypeptides with anti-microbial bioactivity (e.g. coprisin, cathelicidin, LL-37, thuricin CD, lantibiotics), polypeptides with anti-cancer bioactivity (growth inhibiting biologies).

A bacterial cell of the methods and compositions described herein can be any of any species. Preferably, the bacterial cells are of a species and/or strain which is amenable to culture and genetic manipulation. In some embodiments, the bacterial cell can be a gram-positive bacterial cell. In some embodiments, the bacterial cell can be a gram-negative bacterial cell. In some embodiments, the parental strain of the bacterial cell of the technology described herein can be a strain optimized for protein expression. Non-limiting examples of bacterial species and strains suitable for use in the present technologies include Escherichia coli, E. coli BL21, E. coli Tuner, E. coli Rosetta, E. coli JM101, and derivatives of any of the foregoing. Bacterial strains for protein expression are commercially available, e.g. EXPRESS™ Competent E. coli (Cat. No. 02523; New England Biosciences; Ipswich, Mass.). In some embodiments, the cell is an E. coli cell.

In some embodiments, the nucleic acid encoding an engineered CsgA polypeptide is comprised by a cell expressing wild-type CsgA. In some embodiments, the nucleic acid encoding an engineered CsgA polypeptide is comprised by a cell with a mutation and/or deletion of the wild-type CsgA gene, e.g such that the cell does not express wild-type CsgA.

In one aspect, described herein is a biofilm comprising an engineered microbial cell comprising one or more engineered CsgA polypeptide and/or comprising a vector or nucleic acid encoding such a polypeptides. As used herein, a “biofilm” refers to a mass of microorganisms which can adhere or is adhering to a surface. A biofilm comprises a matrix of extracellular polymeric substances, including, but not limited to extracellular DNA, proteins, glyopeptides, and polysaccharides. The nature of a biofilm, such as its structure and composition, can depend on the particular species of bacteria present in the biofilm. Bacteria present in a biofilm are commonly genetically or phenotypically different than corresponding bacteria not in a biofilm, such as isolated bacteria or bacteria in a colony.

In some embodiments, the technology described herein relates to a biofilm that is produced by culturing an engineered microbial cell comprising an engineered CsgA polypeptide (and/or comprising a vector or nucleic acid encoding such a polypeptide) under conditions suitable for the production of a biofilm. Conditions suitable for the production of a biofilm can include, but are not limited to, conditions under which the microbial cell is capable of logarithmic growth and/or polypeptide synthesis. Conditions may vary depending upon the species and strain of microbial cell selected. Conditions for the culture of microbial cells are well known in the art. Biofilm production can also be induced and/or enhanced by methods well known in the art. e.g. contacting cells with subinhibitory concentrations of beta-lactam or aminoglycoside antibiotics, exposing cells to fluid flow, contacting cells with exogenous poly-N-acetylglucosamine (PNAG), or contacting cells with quorum sensing signal molecules. In some embodiments, conditions suitable for the production of a biofilm can also include conditions which increase the expression and secretion of CsgA, e.g. by exogenously expressing CsgD.

In some embodiments, the biofilm can comprise the cell which produced the biofilm. In some embodiments, described herein is a composition comprising an engineered CsgA polypeptide as described herein, e.g., a therapeutic polypeptide fused to CsgA.

When expressed by a cell capable of forming curli, e.g. a cell expressing CsgA, CsgB, CsgC, CsgD, CsgE, CsgF, and CsgG or some subset thereof, CsgA units will be assembled to form curli filaments, e.g. polymeric chains of CsgA. In some embodiments, filaments of the polypeptide can be present in the composition. In some embodiments, the filaments can be part of a proteinaceous network, e.g. multiple filaments which can be, e.g. interwoven, overlapping, and/or in contact with each other. In some embodiments, the proteinaceous network can comprise additional biofilm components, e.g. materials typically found in an E. coli biofilm. Non-limiting examples of biofilm components can include biofilm proteins (e.g. FimA, FimH, Ag43, AidA, and/or TibA) and/or non-proteinaceous biofilm components (e.g. cellulose. PGA and/or colonic acid). In some embodiments, the composition can further comprise an engineered microbial cell comprising an engineered CsgA polypeptide and/or comprising a vector or nucleic acid encoding such a polypeptide.

In one aspect, described herein is the use of a cell, composition, or biofilm comprising an engineered CsgA polypeptide (and/or comprising a vector or nucleic acid encoding such a polypeptide) to display a polypeptide, e.g. within the biofilm, within the composition, and/or on the cell surface. As used herein, “display” refers to expressing the polypeptide (e.g. as an activity polypeptide) in such a manner that it can come in contact with the extracellular environment. A displayed polypeptide can be capable of binding with a binding partner, catalyzing an enzymatic reaction, and/or performing any other activity which it would perform as an isolated polypeptide.

It is contemplated herein that a polypeptide displayed within a biofilm (e.g. an activity polypeptide and/or functionalizing polypeptide) will retain more activity than a soluble version of that polypeptide. It is contemplated herein that a polypeptide displayed within a biofilm (e.g. an activity polypeptide and/or functionalizing polypeptide) will retain more activity than a soluble version of that polypeptide when exposed to activity degrading conditions such as, e.g., high or low pH, organic solvents, desiccation, high or low temperature, radiation, etc.

In one aspect, described herein is the use of a cell, composition, or biofilm comprising an engineered CsgA polypeptide (and/or comprising a vector or nucleic acid encoding such a polypeptide), in an application selected from the group consisting of biocatalysis; industrial biocatalysis; immobilized biocatalysis; chemical production; filtration; isolation of molecules from an aqueous solution; water filtration; bioremediation; nanoparticle synthesis; nanowire synthesis; display of optically active materials; biosensors; surface coating; therapeutic biomaterial; biological scaffold; structural reinforcement of an object; and as a delivery system for therapeutic agents. Exemplary, non-limiting embodiments of such applications and specific activity polypeptides for use therein are described in the Examples herein.

It is contemplated herein that a cell, composition and/or biofilm can comprise multiple different engineered CsgA polypeptides, each of which comprises a different activity polypeptide, e.g. an engineered CsgA polypeptide comprising an enzymatic activity polypeptide and an engineered CsgA polypeptide comprising a binding domain activity polypeptide. A cell, composition, and/or biofilm can comprise one or more engineered CsgA polypeptides, e.g., 1, 2, 3, 4, 5, 6, or more engineered CsgA polypeptides.

In some aspects of the invention, provided herein are pharmaceutical compositions comprising the engineered bacterium disclosed herein, and a pharmaceutically acceptable excipient. In some embodiments, the pharmaceutical composition is formulated for oral administration. In other embodiments, the pharmaceutical composition is formulated for rectal administration. Accordingly, the pharmaceutical compositions contemplated herein may be formulated as a pill, a capsule, a lozenge, or a suppository.

In some aspects, provided herein are methods of producing a recombinant polypeptide, comprising culturing the engineered bacterium provided herein under conditions suitable for expression and export of the recombinant polypeptide from the engineered bacterium. In some embodiments, the recombinant polypeptide comprises at least one CsgA subunit and a therapeutic polypeptide, e.g., an engineered CsgA. In some such embodiments, expression of the recombinant polypeptide is not toxic to the engineered bacterium. Preferably, the level of expression and export of the recombinant polypeptide is maintained, as compared to the level of expression and export of the recombinant polypeptide from an engineered bacterium under the same conditions expressed from a conventional plasmid comprising the heterologous nucleic acid sequence and a selectable marker gene. In some embodiments, the recombinant polypeptide is collected from the cell culture medium comprising the engineered bacterium. In some such embodiments, the engineered bacterium is not exposed to a lysing agent prior to collecting the recombinant protein from the cell culture medium. In some preferred embodiments, the recombinant polypeptide is collected from a supernatant of the cell culture medium. The methods disclosed herein may further comprise purifying the recombinant polypeptide.

In some aspects, disclosed herein are recombinant polypeptides produced using the methods disclosed herein. In other aspects, disclosed herein are biofilms comprising the recombinant polypeptide produced using the methods provided herein.

In other aspects of the invention, provided herein are methods for treating a disease or disorder. Such methods may comprise administering to a subject in need thereof an effective amount of an engineered bacterium provided herein or a pharmaceutical composition provided herein. In some embodiments, the engineered bacterium expresses and exports a recombinant polypeptide comprising at least one CsgA subunit and the therapeutic polypeptide, e.g., an engineered CsgA, thereby treating the disease or disorder.

In some embodiments, the engineered bacterium or the pharmaceutical composition is administered orally. In other embodiments, the engineered bacterium or the pharmaceutical composition is administered rectally. In some preferred embodiments, the subject is a mammal; and most preferably, the mammal is a human.

In some embodiments, the disease or disorder is a gastrointestinal disease or disorder, such as a gastrointestinal disease or disorder selected from the group consisting of inflammatory bowel disease, Crohn's disease, ulcerative colitis, colorectal cancer, ulcer, malabsorption, short-gut syndrome, cul-de-sac syndrome, celiac sprue, tropical sprue, hypogammaglobulinemic sprue, enteritis, short bowel syndrome, and gastrointestinal cancer.

In some such embodiments, the engineered bacterium colonizes the gastrointestinal tract of the subject. Preferably, the engineered bacterium retains the engineered cryptic plasmid for at least 1 to 5 days following administration.

In some aspects of the invention, provided herein are vectors, comprising a cryptic plasmid backbone and a heterologous nucleic acid, e.g., an expression cassette. In some embodiments, the heterologous nucleic acid comprises a nucleic acid sequence encoding csgBACEFG operon, and a nucleic acid sequence encoding a therapeutic polypeptide. In some such vectors, the csgBACEFG operon is derived from E. coli. In some preferred embodiments, the heterologous nucleic acid is inserted into cryptic plasmid pMUT1 backbone or pMUT2 backbone at insertion sites as indicated in Table 3. In some embodiments, the heterologous nucleic acid is inserted within a site amplified by a primer pair comprising the sequences set forth in SEQ ID NOs: 21 and 22; SEQ ID NOs: 23 and 24; SEQ ID NOs: 25 and 26; or SEQ ID NOs: 27 and 28.

The therapeutic polypeptides encoded by the vectors disclosed herein may be selected from the group consisting of an antibody, an antibody fragment, an enzyme, a fusion protein, a hormone, an antigen, a thrombolytic agent, a cytokine, an immunotoxin, and a growth factor. In some embodiments, the therapeutic polypeptide is an antibody fragment, and the antibody fragment is a single chain antibody, such as a nanobody. Such single chain antibodies may be specific for an antigen selected from the group consisting of: carcinogenic embryonic antigen (CEA), glucose transporter 1 (GLUT1), green fluorescent protein (GFP), beta-lactamase, Clostridium difficile Toxin A, Clostridium difficile Toxin B, botulinum toxin (BoTox), cholera toxin (CTX), norovirus capsid protein, rotavirus capsid protein, and Plasmodium membrane protein.

In some preferred embodiments, the therapeutic polypeptide is fused to an amyloid polypeptide. Exemplary amyloid polypeptides may comprise at least one curli subunit.

In certain embodiments, the heterologous nucleic acid further comprises a nucleic acid sequence encoding a polypeptide tag. The polypeptide tag may be selected from the group consisting of a poly-histidine tag, a myc tag a FLAG tag, a hemagglutinin (HA) tag, and a V5 tag.

In some preferred embodiments, the heterologous nucleic acid is operably linked to an inducible promoter. The inducible promoters contemplated herein may be responsive to an inducer selected from the group consisting of IPTG, arabinose, tetracycline, and permissive temperature change. Preferably, the inducible promoter is a temperature sensitive promoter.

In preferred embodiments, the vector backbone is pMUT1 or pMUT2. Such vectors may further comprise a nucleic acid encoding a detectable protein. Exemplary detectable proteins include fluorescent proteins as are known in the art, e.g., green fluorescent protein (GFP) and red fluorescent protein (RFP).

Definitions of common terms in cell biology and molecular biology can be found in The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning. A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); and Current Protocols in Protein Science (CPPS) (John E. Coligan, et. al., ed., John Wiley and Sons, Inc.), which are all incorporated by reference herein in their entireties.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples are set forth as being representative of the present disclosure. These examples are not to be construed as limiting the scope of the present disclosure as these and other equivalent embodiments will be apparent in view of the present disclosure, figures and accompanying claims.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

Unless otherwise stated, the present methods were performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques, Vol. 152, S. L. Berger and A. R. Kimmel Eds., Academic Press Inc., San Diego, USA (1987); and Current Protocols in Protein Science (CPPS) (John E. Coligan, et. al., ed., John Wiley and Sons, Inc.), which are all incorporated by reference herein in their entireties.

### Example 1: Materials and Methods

DNA cloning

All plasmid assembly was performed using Gibson Assembly, with the exception of the pCryptDel #plasmids, where the gRNA array was assembled using Golden Gate assembly due to many repeats in the DNA sequence. Custom DNA oligos were ordered from Integrated DNA Technologies (IDT) and used in PCR with Q5 polymerase (New England Biolabs, USA) to create amplicons for subsequent Gibson assembly. DNA purification from PCR was done with ZymoClean™ Gel DNA Recovery Kit (ZymoGen, USA). DNA assembly products were transformed into chemically competent E. coli Mach1 cells (Thermo Fisher Scientific, USA) and plated onto LB Agar plates with appropriate antibiotics.

DNA libraries were generated by designing degenerate bases at selected locations on DNA oligos, flanked by 25 bp of the unmodified sequence. The resulting DNA was synthesized (IDT) used as primers to make amplicons for plasmid assembly. The resulting pool of assembled plasmid variants was transformed into Mach1 cells and plated onto 10 plates. After overnight incubation at 37° C., the plates were imaged for GFP and RFP fluorescence in a FluorChem™ M Imager (Protein Simple, USA), and colonies were selected.

Colony PCR

Assessment of cryptic plasmids was done by colony PCR using 25 μL reactions with Quick-Load Taq PCR mix (New England Biolabs, USA) following the manufacturer's instructions. After the PCR, the reactions were added to a 1% agarose TAE gel with SybrSafe DNA stain and ran in a gel electrophoresis setup (constant 120V, 35 mins). Gels were then imaged in FluorChem M Imager (Protein Simple).

Bacterial Culture

E. coli bacteria were grown in LB Miller media during plasmid preparation and genetic circuit characterization. For characterization assays, starter cultures of the appropriate bacterial cultures were grown overnight in LB media in a shaking incubator. For all temperature sensitive constructs, started cultures were grown at 30° C., whereas 37° C. was used for all other constructs. Unless explicitly indicated otherwise, all characterization was done at 37° C.

Kinetic plate reader assays were performed by diluting starter culture 1:1000 into the appropriate selective media. Into the wells of black, clear-bottom, 96-well plates (655090, Greiner Bio-One, Germany), 200 μL of the inoculated culture was then added. The plates were then grown in a Synergy HT plate reader (BioTek), reading absorbance (600 nm), GFP (excitation: 485/20 nm, emission: 528/20 nm), RFP (ex: 590/20, em: 645/40). Reads were taken every 10 minutes for 16 hours, and plates were shaken continuously outside of reading (Double Orbital, 548 cpm (2 mm)).

Plasmid Curing

In order to cure Nissle and any derived strains of cryptic plasmids, they were transformed with plasmids pFREE or pCryptDe14.8, in order to cure pMUT1 or pMUT2 respectively. After transformation, cells were grown overnight in liquid LB media with 50 μg/mL kanamycin. Then, the overnight culture was diluted 1:1000 into fresh LB media supplemented with 50 μg/mL kanamycin, 0.2% rhamnose and 0.43 μM anhydrous tetracycline (ATC), and grown overnight at 37° C. After 24 hours, the culture was streaked out onto several LB agar plates without antibiotics and these were left to grow overnight. Then, the colonies were assessed by colony PCR with primers muta5, muta6, muta7 and muta8 to find colonies that had been cured of cryptic plasmids.

Growth and Gene Expression Characterization

Data from kinetic plate reader runs was initially cleaned by subtracting the background signal and smoothing the time courses for all fluorescence and absorbance data. Growth rates were found by fitting the absorbance curves to a Gompertz model, and subsequently extracting the peak growth rate. Promoter strength was quantified from kinetic fluorescence data by first finding the gradient of the fluorescence signal, normalizing this to the absorbance signal, resulting in a per cell measure of fluorescent protein production per unit time. Promoter strength was then quoted to be the average of this term for an hour around peak exponential phase.

Curti Measurement

Curli was measured by the CR method outlined in Kan et al. 39. Bacterial starter cultures were grown overnight in LB and the relevant antibiotics at 30° C. Then, selective LB media was innoculated 1:1000 with starter culture, and placed 300 μL into 1 mL deep well plates (780210, Greiner Bio-One, Germany) in a shaking incubator (900 rpm (1 mm)) at either 30° C. or 37° C. 0.025% CR was added to the media upon inoculation. After 24 hours of growth, 200 μL of each well was transferred into black, clear bottom plates and the absorbance (600 nm) and CR fluorescence (ex: 525 nm, em: 625 nm) was read in a Synergy HT plate reader. The results were then normalized to the host strain without engineered plasmids.

Curli production was also measured by whole cell filtration ELISA to measure the E-tagged CsgA proteins. 80 μL bacterial overnight cultures were added into each well of a 96-well filter plate in triplicate (0.22 μm pore size, Multiscreen-GV, Merck/Millipore Sigma). Samples were vacuum-filtered, and washed in TBST (TBS, 0.1% Tween-20), and blocked for 1.5 hours at 37° C. with 1% bovine serum albumin (BSA) and 0.01% H2O2 in TBST. After additional washing, samples were incubated with HRP-conjugated goat polyclonal E-tag epitope antibody (Novus Biologicals) for 1.5 hours at room temperature (1:5000 in TBST). Following additional washes in TBST, 100 μL Ultra-TMB ELISA reagent (Thermo Scientific) was added to each well and covered with aluminium foil to protect from light. After approximately 15-25 minutes of incubation at room temperature, the reaction was stopped using 50 μL per well of 2 M sulfuric acid. 100 μL were transferred from each well into a 96-well plate and absorbance was measured at 450 nm and 650 nm. The signal was calculated by subtracting the 650 nm absorbance value from the 450 nm absorbance value.

GFP Sequestration Assay

To test the function of the GFP nanobody, bacterial cultures were first grown overnight at stated temperatures in LB media with appropriate antibiotics. They were then pelleted at 3000 g for 10 minutes, washed once in PBS, and resuspended in a solution of PBS containing 4 μg/mL purified sfGFP. The solutions were then left in a rotating mixer for an hour at room temperature, then centrifuged again at 3000 g for 10 minutes. The supernatant GFP signal was then measured in the plate reader, and compared to the fluorescence of the initial sfGFP solution. In order to prevent non-specific GFP protein adsorption to the plasticware used in the experiment, a sterile solution of 1% BSA (bovine serum albumin) in PBS was used to block the plastic tubes prior to the experiment. To do this, the 1.5 mL tubes were filled with 1 mL of the BSA solution and left in a rotating mixer for an hour.

In Vivo Study of Engineered pMUT Plasmid Retention and Protein Expression

The protocol described below was reviewed and approved by the Harvard Medical Area Standing Committee on Animals (HMA IACUC, Ref. No. IS00000516-3). 25 female 8- to 9-week-old C57BL/6NCrl mice were randomly split into five experimental cohorts: WT pMUTs, pM1, pM1-VHH, pM2 and pM2-VHH (N=5). Bacterial suspensions were prepared in advance by growing to mid-exponential phase (OD600 of 0.5) at 30° C. (shaking at 225 RPM), pelleting the cells, resuspending to OD600 of 10 in PBS supplemented with 20% sucrose and 10% glycerol, and flash-freezing in liquid nitrogen. Aliquots of these bacterial suspensions were stored at −80° C. and allowed to thaw immediately preceding daily feeding, in order to maintain consistent bacterial density of the inoculum.

48 hours prior to initial administration of bacteria (day −2), the drinking water was supplemented with 2 g/L carbenicillin (Teknova). Antibiotic-free drinking water was restored 24 hours later (day −1). Starting on day 0, each cohort was fed 50 μL of its respective bacterial suspension by allowing the mice to lap the liquid from a pipette tip (as previously described by Mohawk et al. 48). Bacterial administration was carried out daily from day 0 to day 4. Fecal pellets were collected and weighed daily from day 0 to day 7.

Mice

Female 8- to 9-week-old C57BL/6NCrl mice were obtained from Charles River Laboratories. Mice were housed in sterile vinyl isolators within the Harvard Medical School animal facility, and kept under specific-pathogen-free (SPF) conditions. Both sterile food (JL Rat and Mouse/Auto 6F 5K67, LabDiet) and water were provided ad libitum. All mice were allowed one week to acclimate prior to any experimental procedure. To further minimize impact of living environment on experimental outcomes, mice were randomized between housing isolators at the beginning of the experiment. All experiments were conducted in compliance with the US National Institutes of Health guidelines and approved by the Harvard Medical Area Standing Committee on Animals.

Plasmid Retention Analysis

Immediately following daily collection of fecal pellets, each sample was homogenized in 1 mL of PBS, serially diluted, and plated in quadruplicate to enumerate colony forming units (CFU). Samples were plated on two types of LB agar plates −25 μg/mL chloramphenicol-only plates (Cm) and 100 μg/mL carbenicillin+25 μg/mL chloramphenicol plates (Cm+Carb). While all PBP8-derived strains carried a chromosomal Cm resistance gene, only the engineered pMUT plasmids harbored a Carb resistance marker. Plasmid retention rate was therefore estimated by calculating the Cm+Carb to Cm ratio of sample weight-normalized CFU counts. Following the plating procedure, fecal homogenates were flash-frozen and stored at −80° C. for subsequent analysis.

Fecal Filtration ELISA

To detect E-tagged curli fibers in fecal samples, a filtration ELISA protocol was adapted from Praveschotinunt et al. 35. Fecal homogenate was centrifuged at 1000 g for 1 minute to separate large insoluble material, and the supernatant was transferred onto a 96-well filter plate in triplicate (0.22 μm pore size, Multiscreen-GV, Merck/Millipore Sigma). For each sample, the homogenate volume dispensed was normalized to 1.25 mg of fecal pellet per well. After samples were added to the filter plate, the procedure to detect E-tagged material proceeded as described above in the curli measurement section. In each assay, the signal was normalized by dividing by the WT pMUTs control, such that the WT pMUTs control corresponded to 100%.

### Example 2: Revision of the pMUT Cryptic Plasmid Sequences

EcN's cryptic plasmids were first documented by Hacker et al. in 2002 ²⁰, who published the sequences for pMUT1 and pMUT2 on the National Center for Biotechnology Information (NCBI) database with accession numbers A84793 and A95448 respectively. Since then, 3 whole genome-sequencing projects for EcN have been uploaded to NCBI, with 2 fully assembled genomes. The first assembly, ASM71459v1 (Reister et al.²¹), resulted in a single sequence containing the chromosome and both plasmids, with the plasmids erroneously inserted multiple times within the chromosomal sequence. A later assembly, ASM354697v1, has a genomic sequence separate from the 2 cryptic plasmid sequences (labelled pNissle1 and pMUT2). Here, the pNissle1 sequence contains both the sequence for pMUT1 and pMUT2 and is likely also an erroneous assembly. Also, the pMUT sequences from the whole genome sequencing projects differed from those originally uploaded, A84793 and A95448, which were sequenced using Sanger sequencing. As such, a correct pMUT1 sequence on NCBI could not be found, one was thus uploaded one for reference, NCBI submission ID 2292834, and NCBI accession CP023342 is referred to for the correct pMUT2 sequence, each of which are incorporated by reference herein in their entirety. The correct pMUT sequences were confirmed by Sanger sequencing backbones of the pMUT-derived engineered vectors, finding the sequence traces aligned exactly with those derived from the whole genome sequencing efforts.

TABLE 1
Escherichia coli Nissle 1917 plasmid pMUT1, complete sequence.
NCBI submission ID 2292834/ ACCESSION# MW240712 (SEQ ID NO: 1)
Bases 1 to 3173

```
      1 gtttcagtgg tgcgtacaat taagggatta tggtaaatat atgagctatg cgataacttt
    61 aactgtgaag cgatgaaccc attacaggca aagccaatta ctcctgacag tggtttagcc
  121 agaagcaggg ctaccaagac caatgcaata agtaatatat cgttttgcta tcgtgcatcg
  181 ctcgcgctca gttccattgt gcttttttaa gctgtcgttt ttcttacggt atataccggt
  241 tttttatggc gtggtttctt aacttgttca gctactgatg gacccattgt atctaggtag
  301 tcaactagct ttgttagatc ataaatattg cgaccaccat atcggcgatc actcttcgat
  361 gttggtttct tgtccaagag attagctttt tcaagatcat tgatagctct ctgaacagtc
  421 cgtacagaaa cccccatacg tatggctaga ctttccattg acggatgcgg ccactcttgc
  481 aaactccacc agtgaacgat caggttaagt agtgtgttaa aggccactga agttagcttt
  541 ttctcgtttt gtataaaaaa caatacggta ggcactgctg tccagccaag agacaaaccg
  601 ccagctttcc atttattctt aacggagtaa gtcattgatt ttcctaagcc ccaaaatatt
  661 taaagtatat attatatggt atattcatat gaatagggtg acactggcgc cattattgtg
  721 caaccaaaaa agactactct gaaaacgagg aaaagatttt ttcctgcctg aattagatac
  781 ggagttagcg atatgaaaac cgaacaacgt catgatcttg ttaaagatat tgaggttttt
  841 ggcgtatcct tgtctctgtt gatttccaga gcgaatgaga agtctgttac aatgccatct
  901 ggtctaagtc gggagcagag aagagcatgg gcagcggagc aggcgcgcaa aatccacaat
  961 tgaatattgt ctcattctct gagaccttca acctttatta cacatccaga tattctgcaa
1021 aaacactcga taaaatcgat gatttcattg agcattttga aaaatacaat ctctttggcg
1081 atcctttaaa aggatatcca gcttggactg gcaaagtatc gccatcgtgg aaagtgcctg
1141 atcattacga aaacaaagaa gctattgaga agtatgctag agctaacaaa ttatggcatg
1201 ctcatttagg cgatccggtt tttaaagata cgtttcatgg gaaatacaag gtttctgact
1261 gggttattca tttccagcgg ctgacaccga accatataaa gcttttagag cttggatacc
1321 atgacccaat gaagctacct caaaactttg aatgatcgag cggcaggcta aatgaaatct
1381 tgagattcat tcagtctcgt cgtaatctca ctattgtaaa aacgaaaaaa ccaccctggc
1441 aggtggtttt tcgaaggtta gttaatcctg gcagattctc taaccgtggt aacagtcttg
1501 tgcgagacat gtcaccaaat actgtccttt cagtgtagcc tcagttaggc cgccacttca
1561 agaactctcg ttacatctct cgcacatcct gcttaccagt ggccgttgcc agtggcgtta
1621 agtcgtgtct taccgggttg gactcaagac gatagttacc ggataaggcg cagcggtcgg
1681 gctgaacggg gggttcgtgc acacagccca gcttggagcg aacgacctac accgaaccga
1741 gataccaaca gcgtgagcta tgagaaagcg ccacgcttcc cgaagggaga aaggcggaca
1801 ggtatccggt aagcggcagg gtcggaacag gagagcgcac gagggagctt ccagggggaa
1861 acgcctggta tctttatagt cctgtcgggt ttcgccacct ctgacttgag cgtcgatttt
1921 tgtgatgctc gtcagggggg cggagcctat ggaaaaacgc ctcccgcgcg cccttcttct
1981 gggtctttgt cttttgctca catgttcttt ccggttttat cccccgattc tgtggataac
2041 cgtattaccg cctttgagtg agctgacacc gctcgccgca gtcgaacgac cgagcgtagc
2101 gagtcagtga gcgaggaagc ggaagagcgc cttatgtgac attttctcct tacgctcctc
2161 tatgccgttc tgcatcctgt ccggatgcgt tatatcccgg taagattttc cgcttcaaag
2221 cgtgtctgta tgctgttctg gagttcttct gcgagttcgt gcagtttctc acacatggcg
2281 gcctgttcgt cggcattgag tgcgtccagt ttttcgagca gcgtcaggct ctgacttttt
2341 atgaatcccg ccatgttgag tacggcttgc tgctgcttat tcatcttttc gttttctccg
2401 ttctgtctgt catctgcgtt gtgtgattat atcgcgtacc acttttcgac tgttttgctg
2461 ccgctattct gccgcttggc tttttgacgg gcatttctgt cagacaacac tgtcactgcc
2521 aaaaaactgc cgtgcctttg tcggtaattc gagcttgctg acaggacagg atgtgcaatt
2581 gttataccgc gcatacatgc acgctattac aattgccctg gtcagggctt tgccccgaca
2641 cccatgtcag atacggagcc atgttttatg acaaaacgaa gtggaagtaa tacgcgcagg
2701 cgggctatca gtcgccctgt tcgtctgacg gcagaagaag accaggaaat cagaaaaagg
2761 gctgctgaat gcggcaagac cgtttccggt tttttacggg cggcagctct cggtaagaaa
2821 gttaactcac tgactgatga tcgggtactg aaagaagtta tgagactggg ggcgttacag
2881 aaaaaactct ttatcgacgg caagcgtgtc ggggacaggg agtatgcgga ggtgctgatc
2941 gctattacgg agtatcaccg tgccctgtta tccaggctta tggcagatta gcttcccgga
3001 gagaaaactg tcgaaaactg acggtatgaa caccgtaagc tcccaaagtg atcaccattc
3061 gctttcatgc atagctatgc agcgagctga aaacgatcct gacgcatcct tcctgttttt
3121 ccggggtaaa acatctcttt ttgcggtgtc tgcgtcagaa tcgcgttcag cgc
```

TABLE 2
Escherichia coli Nissle 1917 plasmid pMUT2, complete sequence
ACCESSION# CP023342 (SEQ ID NO: 2)
Bases 1 to 5514

```
      1 gcatgctcaa ggcctgacaa accctgtcgt ttttcgccaa ctcctgcgag gtaacctcga
    61 acatgcgctg taagttggcg tagctgtcct gccacgcttg ctgctgttgt tcgtagtgcc
  121 tctgtaagct ctctaatgcg ctcagaagct gctgctccat ttcggtcatg aatctcttca
  181 ccctgataga taaaaccgcc cagaatcgat tctgtggcgt ctgatgaggt tatttggcgc
  241 tgtacttgat gacctgacga tgttgagcgt tcttgtactc gtcgatcttc ttcgccccct
  301 gcggaaggat caggtaatac acgctcttgt tcttggaatc gtgaattatc gatacgccgg
  361 ctccggtctg gctctttaag tcctgcagga tctggctctg ctcgctgatt tcgttctggc
  421 gttcgaccac gatagtcccg agataccaag ctactccaat caatatcgca aacaggatcc
  481 cacttaatgc caggctgtac agccatgtca ttccgactaa gcggtgtatc tgttttagct
  541 ggctgtcgtt ctcttcttgg atagcggtct gtatgttccc tgagcttaat ttcaaggcct
  601 cggtgatacg tttctcgtgc ttctcgaatg cgttcgcgac gctcgttgcg gtagtcttgg
  661 cttgctgctt cgatttgctc tcgaactccc gcgctaaatt taaaatctcg ctcatacagc
  721 actcctttta agcgaatatt cgggccacct gccggatcag caatactgat actggatttg
  781 gtttcccgta cgaccgacaa tccggcatcg gtaaggtggg aaatcacccc tttacgatcc
  841 gtaatttctc cctgctcaat caagctgatt agccctttgg taatggcttc cgctgcctgc
  901 tgtttgttgc gaggaaggtc attagagggg gttaatgctc ggcgattagc agggtcattc
  961 gggtcgcgta acccaagccg gtcattggtg agggtttgcc atgcgttaac acgaggccgg
1021 tcagcccgat caaagtaagg ttgtagccgt tttccgctct gcaattcgat gttcgggata
1081 acaaaattca attcaagacg ccctttgtcc tgatgttgaa cccagaggca ggcatactgg
1141 tctttatcta gaccggtcat caatgtctgc tcccattcat ccatcaatcg ctgcttttcg
1201 ccttcgggta aatcactctc ctgaaaagag agcacgccag aggtataagt tcgggcaaat
1261 tcgcagccat caatcagctc tttgacgtgc tcagggttac cccgtaacac cgtcgcttgt
1321 tcgcgctgac gatcagggcc cagaaggtaa tcgacaggac cactcccgcc accggcacca
1381 cgaccatgaa tcttaacgat cacgatgttg ctccagcagt tcggcaagat gttggtcaat
1441 gctattgagc accgctaaca acgacacccg ttcttgcggc ggtaagccat gctgattcaa
1501 gtaacgggct atttgattga ggttattacc gatcccgctg acctgacgta acaaggtcgg
1561 gtctacggta ggtagcggac gacgccgagc tgtacgcgat tcgcctaagc caacggctcg
1621 taaccactcg gccaaatgct tacggtcaca gcgttcaagt agccgctgat gctccgcttc
1681 ggtgagtctg attttgatct ctttggtgcg cttttccatg agaatccgct gagaaagttt
1741 cgcacccaaa gtgcgaattt tcgcagtgga tgcaaggggt ttcggggggc ggcgagcccc
1801 ctgaaacagt cacagacggc acctcgaaga gGGGACGCTG TGTGTACTgg cttagtacag
1861 catctatcgt acatcgaggt cgcatcacgc acaaacaaaa agccccgcaa aagcagggct
1921 gttatctgat ataggttgtt ttgtctcaca cggcagcgga agaggaatcc gaagtggtac
1981 tggtagtagt attggatgct gctgacgata ttttccgctt tgacccaagg cttaaataat
2041 caatgcctgt aatcaacgat ctcaatacgc cttcggatac catagcgata aacgtatctt
2101 gctggttatg gcttgcgatg caaatcgtag catcaccttt tttatacttt aaaacacctg
2161 ctaaatatcc attttcatct agaacactct taagatgttc atttgttatt gtttgtagcg
2221 tttgctttgt ttcgcttcga gcatacgcct tagctagctt ccgagaaaaa gcatccgcat
2281 catgactatc tttatttact cgctcaataa atttgcttaa gtcaacaaat cccttaaaac
2341 gagtggacat attgttaaca aaatcagtgg cagcattttt tatccatgct ttatagccaa
2401 aaaaacgctc gaaaacattt tggtcgtaga taaataccgt atcgccagca aaaacaagag
2461 atgccttacc atcaatagaa atcatatctt gatctactcg agatagttct tttttgctaa
2521 acccataaaa attatttttc tttcttgata agttttgaac tggatattgc ttcttgtata
2581 tggttataca attgtcgtca ttattcttac tcaaaacgaa aatgattgag tcaacttttg
2641 atattagatc cacactgtca aaatcaacaa ttgggatatt ttcattgcca tcaccaccaa
2701 acccagcatc aaacacaatg ccagtcagaa acttcatttg ctcattatca tactgctcag
2761 catcatttaa aaatgaaatg gtgttggtgc ggtcacttag tgtattaaca tctgatacta
2821 tcacaacccg attttctaaa tcagatatag ttttctttat cacattatca ataatggact
2881 gttttagctc actgtcattt ttaaggatgg caattttata gctaaaagag tccttagcac
2941 ccgctttacc tttattttta aagttaaagt aagtgtgcaa tgtaacatcg ttaatatcac
3001 aatcaaaatg cttatacagt tctaaaagct cttgtgcttt ttcttcatta tgctccaaag
3061 catcaagatc taaggcatcg tcactcatca tcattcctct atgatttttt tcgcgaacgt
3121 taaataatca tcatgattta taactctgat aaaatcattt tcttttatta aatctttaga
3181 taaaactatc aactcaccgt cttgcgtttt ttcccttcca ttagctacca cactgtaagt
3241 aatcttaTAG GCAGAAACat taaataatga caatgttggg ttgcagtgaa ttctttttgt
3301 tttgatgtgc aaaaaaccga cgataatcaa aacaaacaaa aaattaacta tatttgatgg
3361 tttgcttaaa tcagtaaaga ccaacggcat tatgtacgtt gataaaaaag aaagatactc
3421 accggattct tttttacatg aaacgacctt taactttctt gacaccgcac cggagtctaa
3481 gtttttcaaa acccatcgat accaaatgta tgtataagaa caagttaaaa tcaaagcccc
3541 gcagatcact gacctcaata cagaaaatgt taatctgcta tttgaatagt cgagtacgca
3601 ttgaaatttt ccatccgcgc cagaacacga agacatggcc ttatctaaaa cagaccatac
3661 gttatcaata ccagaaaaat atattgttat cggtataaat aaaaacaaca ttgataagag
3721 atacattcta attttcattt ttgtaaaatt tcctgtacca cgttgatcta cttattccta
3781 aagaaatcca ttctccatct ctaactttcg gccttccacc accagagctt ttttttccac
3841 gttgacgctg aatttcagaa gtatgtgttt gtttaacata ctcttcaaag ccaagctctg
3901 taaggttctt acttgtccac ttagccacac ttttagcaat tcccatgact tcgttctcgt
3961 ctaaaggtgc ggagaactgc aggttgtagg ctttagcgcg ttcaatgcag gcttgtagcc
4021 attggtcata ctgcggccag ccttggcgga tagcgcggta agcccacttg cgggttttat
4081 cgaagagggt gcagttacgg cctaaaccgt agtccggcag gatttcgcgg tcattggctg
4141 cgccaaggtc gaggtaatcg gctaaccagt caagggtata gagctctggc tgccagacgg
4201 tgatctgcca gtgcaggtgg ttcggattct tgcaaattag ccctgaatac cccgcatctg
4261 cgcccaattt tttacgcagc gcattctcga tggcggcggc gtatttaagg ggggcagctc
4321 gaccatccgg cgcggtacgt accgccgtat gcaaggcata caacaggtga gcatgtccgt
4381 tctcggggtt tttgatggtg agtgtgggcg caggtgcccc cagatcggcc caatcaatcg
4441 cggctccggc tctgtccacg tcaaagcaaa gccagtacat ggcgtgaggc tgattaaact
4501 ggatgtattt tgcgaggaga gcacgctctt taccggcaat gcgaacacca aactgtaaat
4561 catcggagaa gtacggcttg tggggtaacc ggtcgttaaa aagcgttaaa gcctgattat
4621 ccaaggctcc cagccttatg gcggggctgt tgttttgcac gctgcatgtg ctaatatcct
4681 ttctaggttt cgacctagcc ctgaatgtca tgtccgctcg ccaaagtaga gcatgatttc
4741 ggGGCTTTGT TTTTTCTGcc actaagttac acctcaacaa cggtttttgt catccccgac
4801 aatccgttat tcctgcttgt tctcgcacgg ctttacgctc atactacttc ttgtagatac
4861 acttgtcact acatcaagag gtgagatgat ggccacgatt aatattcgga tcgatgacga
4921 gctgaaaagc cgctcttatg ccgcactgga aaagctgggc gtaacgccgt ccgaggttct
4981 gcgccaaaca ctggaatatg tggcccaaag cggacgtttg ccgttccagc aggttttgct
5041 gaccgaggat gatgccgatt tgatggctat cgttcgggat cgtctggaaa acccacaggc
5101 gggcgtaaag gtgtcactgg atgagctata accttgaatt tgatccccga gccctgaagg
5161 aatggcgcaa gctcggggat gatgtccgtc tgcagttcaa gaaaaaactc gagcaggttc
5221 tacaacaccc gcggatcgat aaaaatcgcc tgcgagagct gcatgactgc tacaaaatca
5281 agctccgtgc atccggttat cgcttggtct atcaggttcg cgatcaaacc attacggtat
5341 tcgtggtggc ggtcggtaag cgcgagcgtt ctgccgctta cgatgcggcc gataaacgct
5401 tataaactca tgccgtgacc gcgagaatac cgctgttcgt gccgcttggc taattgctca
5461 agcggcgcac tgttctgttt aagctctcga cttgctgcgc caagccggtg actt
```

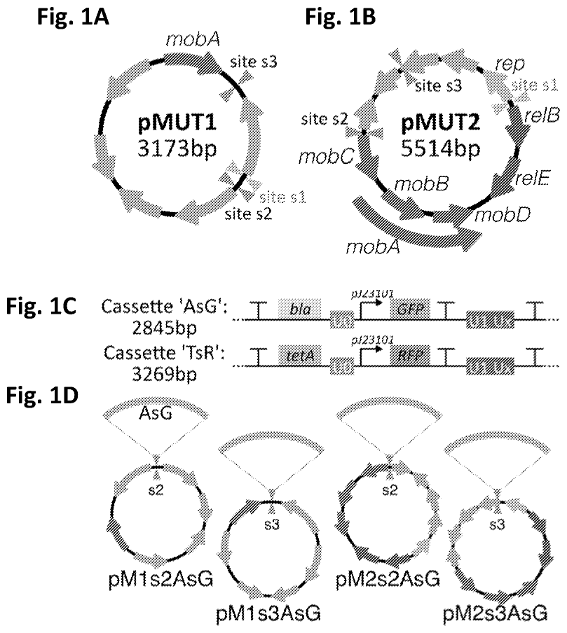

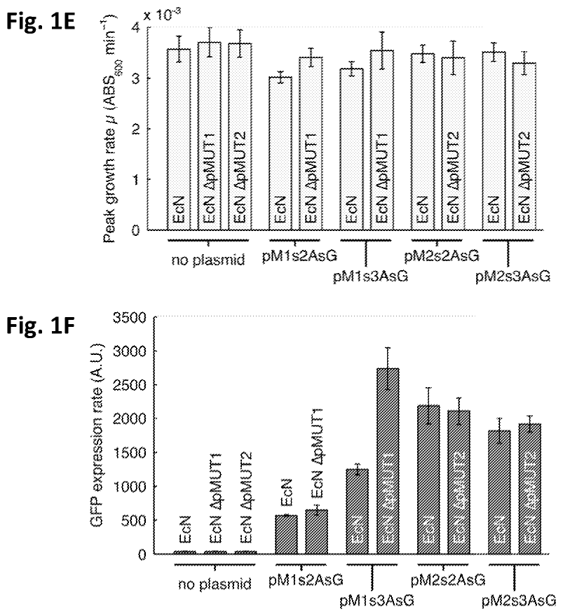

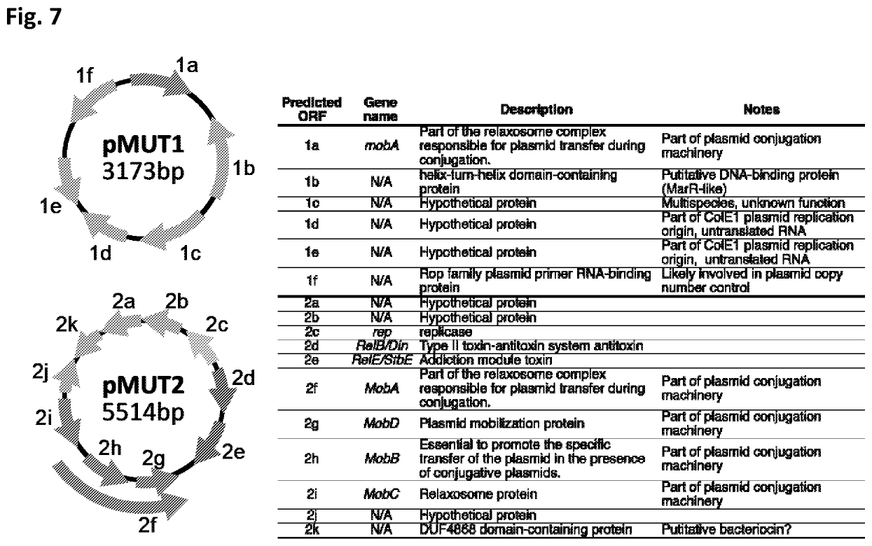

FIG. 1A shows the plasmid maps and lengths of pMUT1 and pMUT2, and FIG. 7 shows the annotations as determined by the NCBI automated prokaryotic annotation pipeline 22 in greater detail. pMUT1 has a typical ColE1 origin of replication 17. pMUT2 is 96.9% homologous to the pUB6060 plasmid from Plesiomonas shigelloides, which has been described as having a ColE2-like replication and ColE1-like mobilization loci 23. Both plasmids contain mob genes involved in plasmid transfer, however both plasmids lack the full gene complement necessary for conjugation, and have previously been described as non-transferable 24. Most of the putative proteins found on the plasmids have no known functions (FIG. 7), except for the relB-relE toxin-antitoxin system on pMUT2. Toxin-antitoxin systems, often found on plasmids, are known to promote plasmid maintenance ²⁶, so it is likely that these genes contribute to pMUT2 stability in EcN.

### Example 3: Engineering the pMUT Plasmids

pMUT engineering began by selecting 3 sites, s1-s3, (FIG. 1A-1B) on each plasmid to insert an insulated cassette encoding antibiotic resistance and a fluorescent protein. The selected sites did not contain any known proteins and were away from the origin of replication in order to avoid disrupting any native functions. The entire sequence for each cryptic plasmid was kept as vector backbone, in order to maintain any features that may contribute to the stability of the plasmids. The cryptic plasmids were amplified by PCR with primers (Table 5) to act as the vector backbone onto which the cassette was inserted. For both pMUT1 and pMUT2, cassette insertion at 3 sites were tested. Notably, a plasmid could not be assembled for insertion site 1(s1) as the backbone could not be amplified by PCR despite trying two primer pair variants.

TABLE 3
Insertion site sequences

| Cryptic | | | Insertion |
|---|---|---|---|
| Plasmid | Site | Sequence | site* |
| pMUT1 | s1 | attatatggtatattcataT\| | 691 |
| | | Gaatagggtgacactggcgc | |
| | | (SEQ ID NO: 3) | |
| pMUT1 | s2 | attatatggtatattcataT\| | 781 |
| | | Gaatagggtgacactggcgc | |
| | | (SEQ ID NO: 4) | |
| pMUT1 | s3 | gtcagaatcgcgttcagcgC\| | 0 |
| | | Gtttcagtggtgcgtacaat | |
| | | (SEQ ID NO: 5) | |
| pMUT2 | s1 | tcggggctttgttttttctG\| | 4759 |
| | | Ccactaagttacacctcaac | |
| | | (SEQ ID NO: 6) | |
| pMUT2 | s2 | gaggggacgctgtgtgtacT\| | 1849 |
| | | Ggcttagtacagcatctatc | |
| | | (SEQ ID NO: 7) | |
| pMUT2 | s3 | taatcttataggcagaaaC\| | 3259 |
| | | Attaaataatgacaatgttg | |
| | | (SEQ ID NO: 8) | |

Vertical line indicates position of cassette insertion within pMUT1 and pMUT2 sequence.
*Nucleotide number in either pMUTI or pMUT2 where insertion occurs.

The 2 successful insertion cassettes for gene expression were tested (FIG. 1C): ‘AsG’ which contained an ampicillin resistance gene (bla) and constitutively expressed superfolder GFP; and ‘TsR’, which contained a tetracycline resistance gene (tetA) and a constitutively expressed mCherry RFP. Both of these transcriptional elements were flanked by terminators to insulate the insertion cassette from transcription on the plasmid backbone and vice-versa. Furthermore, Universal Nucleotide Sequences (UNS) from Torella et al. ²⁷ were added to the cassettes in order to allow for the rapid assembly of modular genetic elements. The UNS sequences are 40 bp genetic segments that act as spacer elements without significant DNA structure or function. UNS sequences flanked each functional module created, and are labelled as ‘U #’ in genetic circuit diagrams, and the UNS sequence information can be found in Table 4.

TABLE 4
UNS sequences used in the engineered
pMUT plasmid designs.

| | | SEQ |
|---|---|---|
| | | ID |
| Name | Sequence | NO: |
| UNS0 | GTTCCTTATCATCTGGCGAA | 9 |
| | TCGGACCCACAAGAGCACTG | |
| UNS1 | CATTACTCGCATCCATTCTC | 10 |
| | AGGCTGTCTCGTCTCGTCTC | |
| UNS2 | GCTGGGAGTTCGTAGACGGA | 11 |
| | AACAAACGCAGAATCCAAGC | |
| UNS3 | GCACTGAAGGTCCTCAATCG | 12 |
| | CACTGGAAACATCAAGGTCG | |
| UNS4 | CTGACCTCCTGCCAGCAATA | 13 |
| | GTAAGACAACACGCAAAGTC | |
| UNS5 | GAGCCAACTCCCTTTACAAC | 14 |
| | CTCACTCAAGTCCGTTAGAG | |
| UNS6 | CTCGTTCGCTGCCACCTAAG | 15 |
| | AATACTCTACGGTCACATAC | |
| UNSX | CCAGGATACATAGATTACCA | 16 |
| | CAACTCCGAGCCCTTCCACC | |

Inserts ‘AsG’ and ‘TsR’ were cloned into the sites on either pMUT1 or pMUT2 to obtain plasmids pMXsYAsG and pMXsYTsR, where X is either 1 or 2, referring to pMUT1 or pMUT2, and where sY is the insertion site number (FIG. 1D). The gene expression from each insertion site was tested by measuring the cell density and GFP fluorescence in a kinetic run for each insertion site with plasmids pMXsYAsG. These assays were performed with both unmodified EcN transformed with the engineered pMUT plasmid, as well as with transformed EcN where the corresponding native pMUT had been knocked out.

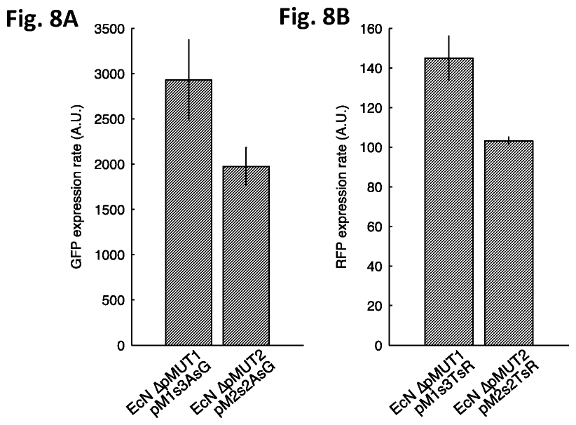

As reported before 18, EcN pMUT plasmid knockouts did not grow differently under lab conditions (FIG. 1E). Growth rates were broadly similar in all cases with the engineered plasmids, although in the pMUT1 derived vectors, the presence of the native pMUT1 reduced growth slightly (p<0.001 in both cases). The insertion sites were found to influence GFP expression levels, with site 3 on pMUT1 and site 2 on pMUT2 giving the highest GFP expression levels (FIG. 1F). The expression rates from pM1s2AsG further showed that the presence of the native plasmid altered gene expression from the engineered plasmid significantly. Engineered vectors capable of high levels of gene expression were desired, thus the high performing sites pM1s3 and pM2s2 were selected for further use. RFP expression using pM1s3TsR and pM2s2TsR was also characterized, finding a similar ratio of gene expression strengths to the GFP data (FIG. 8A-8B), suggesting that their relative rate of gene expression is independent of the protein expressed.

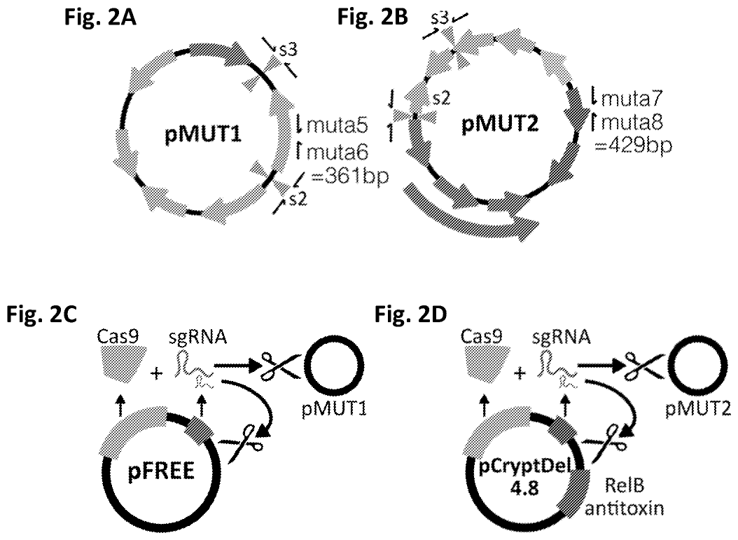

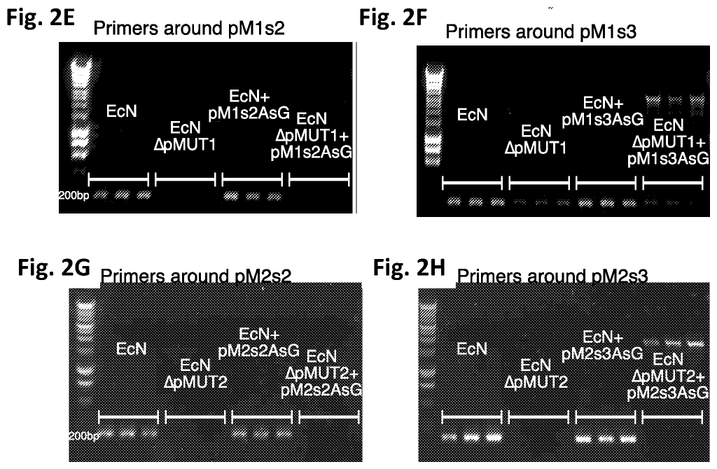

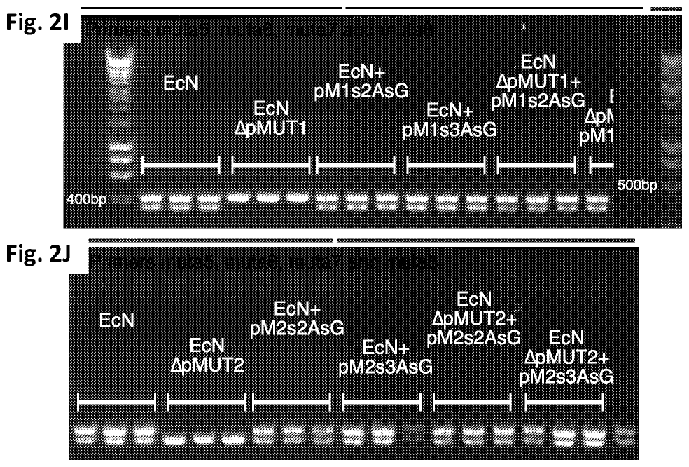

It was found that transforming with the engineered plasmids did not displace the native plasmids. The pMUT plasmids were tested for with DNA primers muta5, muta6, muta7, and muta8 (Table 5), developed by Blum-Oehler et al. ¹⁷ to detect pMUT1 and pMUT2 in clinical samples. In a multiplex PCR with these 4 muta primers, a 361 bp product was formed when pMUT1 was present, and a 429 bp product when pMUT2 was present (FIG. 2A-2B).

TABLE 5
Primers used to assess pMUTs in colony PCR, as well as the primers
used to insert there combinant cassettes onto the pMUT plasmids
to make the engineered versions. In all cases, the
capitalized sequences indicate homology to the pMUT target. Primer
pairs in italic could not produce an amplicon with EcN DNA as a template.

| Primer Name | Sequence | Target | Seq ID NO: |
|---|---|---|---|
| Muta 5 | AACTGTGAAGCGATGAACCC | pMUT1 | 17 |
| Muta 6 | GGACTGTTCAGAGAGCTATC | pMUT1 | 18 |
| Muta 7 | GACCAAGCGATAACCGGATG | pMUT2 | 19 |
| Muta 8 | GTGAGATGATGGCCACGATT | pMUT2 | 20 |
| pM1S2chk_F | GAATAGGGTGACACTGGCGCC | pMUT1 | 21 |
| pM1S2chk_R | CCAGATGGCATTGTAACAGACTTCTC | pMUT1 | 22 |
| pM1S3chk_F | CGCATCCTTCCTGTTTTTCCGG | pMUT1 | 23 |
| pM1S3chk_R | GTCTTGGTAGCCCTGCTTCTGG | pMUT1 | 24 |
| pM2S2chk_F | AGTTTCGCACCCAAAGTGCG | pMUT2 | 25 |
| pM2S2chk_R | GACAAAACAACCTATATCAGATAACAGC | pMUT2 | 26 |
| pM2S3chk_F | GATAAAACTATCAACTCACCGTCTTG | pMUT2 | 27 |
| pM2S3chk_R | GCCGTTGGTCTTTACTGATTTAAG | pMUT2 | 28 |
| M1_1F | aaaccttgcttcttcgcggtGAATAGGGTGACACTGGCG | pMUT1 | 29 |
| M1_1R | accgcattctagatttagggATATGAATATACCATATAA | pMUT1 | 30 |
| | TATATACTTTAAATATTTTGG | | |
| M1_1F2 | aaaccttgcttcttcgcggtGAATAGGGTGACACTGGCGCCATTATTGTG | pMUT1 | 31 |
| M1_1R2 | accgcattctagatttagggATATGAATATACCATATAATATATACTTTA | pMUT1 | 32 |
| | AATATTTTGGGGCTTAG | | |
| M1_2F | aaaccttgcttcttcgcggtGGAGTTAGCGATATGAAAACCGAACAACG | pMUT1 | 33 |
| M1_2R | accgcattctagatttagggGTATCTAATTCAGGCAGGAAAAAATCTTT | pMUT1 | 34 |
| | TCC | | |
| M1_3F | aaaccttgcttcttcgcggtGTTTCAGTGGTGCGTACAATTAAG | pMUT1 | 35 |
| M1_3R | accgcattctagatttagggGCGCTGAACGCGATTCTG | pMUT1 | 36 |
| M2_1F | aaaccttgcttcttcgcggtCCACTAAGTTACACCTCAACAACG | pMUT2 | 37 |
| M2_1R | accgcattctagatttagggCAGAAAAAACAAAGCCCCG | pMUT2 | 38 |
| M2_1F2 | aaaccttgcttcttcgcggtCCACTAAGTTACACCTCAACAACGG | pMUT2 | 39 |
| M2_1R2 | accgcattctagatttagggCAGAAAAAACAAAGCCCCGAAATCATGC | pMUT2 | 40 |
| M2_2F | aaaccttgcttcttcgcggtCCACTAAGTTACACCTCAACAACGG | pMUT2 | 41 |
| M2_2R | accgcattctagatttagggCAGAAAAAACAAAGCCCCGAAATCATGC | pMUT2 | 42 |
| M2_3F | aaaccttgcttcttcgcggtATTAAATAATGACAATGTTGGGTTG | pMUT2 | 43 |
| M2_3R | accgcattctagatttagggGTTTCTGCCTATAAGATTACTTACAGTG | pMUT2 | 44 |

Furthermore, primers were designed around the insertion sites on pMUT1 and pMUT2 to distinguish the native and engineered pMUT plasmids. Colonies were expected in which the native pMUTs were knocked out through plasmid incompatibility—a process whereby two plasmids cannot stably coexist in the same bacterial cell line over multiple generations, typically occurring in plasmids containing similar or identical replication mechanisms. However, when unmodified EcN was transformed with an ampicillin resistant engineered pMUT plasmid (pM1s3AsG or pM2s2AsG), and grown on selective media, colony PCR with primers around the insertion sites (primers pMXsY_chk_F and R) produced a short 200 bp product, indicating the presence of native plasmid (FIG. 2E-2H). This was despite the native and engineered plasmids having identical origins, and only the engineered plasmids conferred antibiotic resistance. Since the data indicated that unmodified pMUT plasmid can impact the performance of the engineered plasmids, a technique was developed to rapidly remove the native plasmids prior to transformation with engineered pMUTs.

### Example 4: Curing the Native pMUT Plasmids

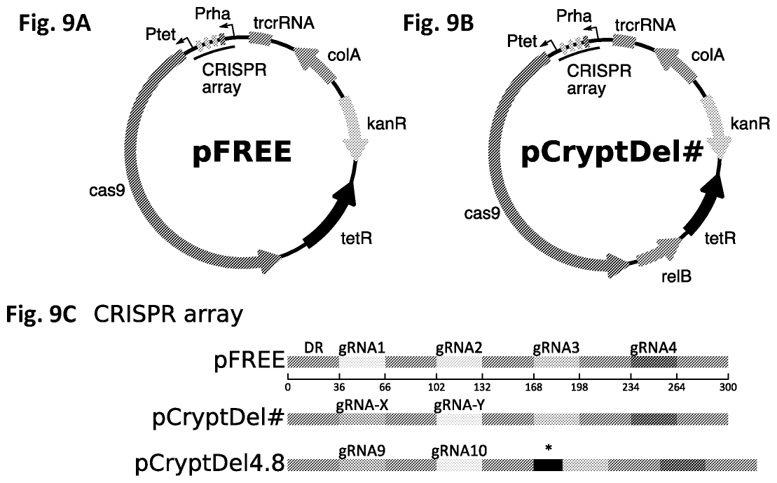

Since transforming EcN with engineered pMUTs did not displace the native plasmids, a strategy was required to remove them. Whilst pMUT curing strategies exist, they rely on plasmid incompatibility to knock out native plasmids ¹⁸, which data indicates is not an immediate process and requires multiple weeks of streaking onto selective media. Therefore a CRISPR-Cas9 strategy was used to cleave both native pMUT plasmids, based on the pFREE system of Lauritsen et al. ²⁸. The pFREE plasmid (FIG. 9A) contains a Cas9 protein and 4 guide RNAs (gRNAs) that target two sites on two common origins of replication, ColE1 and pSC101, in order to rapidly cleave and cure plasmids with those origins. The pFREE plasmid also harbours a variant of the ColE1 origin, and therefore also cures itself during this process.

It was found that the pFREE plasmid cured pMUT1 (FIG. 2C) with a success rate of around 60% (43 out of 73 colonies tested), but unsurprisingly did not cure pMUT2 as it does not have a ColE1 origin. To cure pMUT2, guide RNAs were redesigned to target locations on pMUT2 (Table 6) to make the pCryptDel range of plasmids.

TABLE 6
gRNA sequences used in the pFREE and pCryptDel plasmids. gRNAs 1-4
(starred) were found in the original pFREE sequence from Lauritsen et al. [1].
In each case, the gRNA name, sequence and target are shown.

| gRNA | | | |
|---|---|---|---|
| Name | Sequence | Target | Seq ID NO: |
| gRNA1* | atgaactagcgattagtcgctatgacttaa | Targets pSC101 origin | 45 |
| gRNA2* | aaccacactagagaacatactggctaaata | Targets pSC101 origin | 46 |
| gRNA3* | ggttggactcaagacgatagttaccggata | Targets ColE1-like origins | 47 |
| | | except colA | |
| gRNA4* | ggogaaacccgacaggactataaagatacc | Targets ColE1-like origins | 48 |
| | | including colA | |
| gRNA5 | ccgatttgatggctatcgttcgggatcgtc | Targets pMUT2 at RelE | 49 |
| gRNA6 | aactgcaccctcttcgataaaacccgcaag | Targets pMUT2 at hypothetical | 50 |
| | | protein | |
| gRNA7 | gctctcttttcaggagagtgatttacccga | Targets pMUT2 at relaxase | 51 |
| gRNA8 | ttgattttgtagcagtcatgcagctctcgc | Targets pMUT2 at RelB | 52 |
| gRNA9 | cttgaatttgatccccgagccctgaaggaa | Targets pMUT2 at RelB | 53 |
| gRNA10 | gcccacactcaccatcaaaaaccccgagaa | Targets pMUT2 at relaxase | 54 |

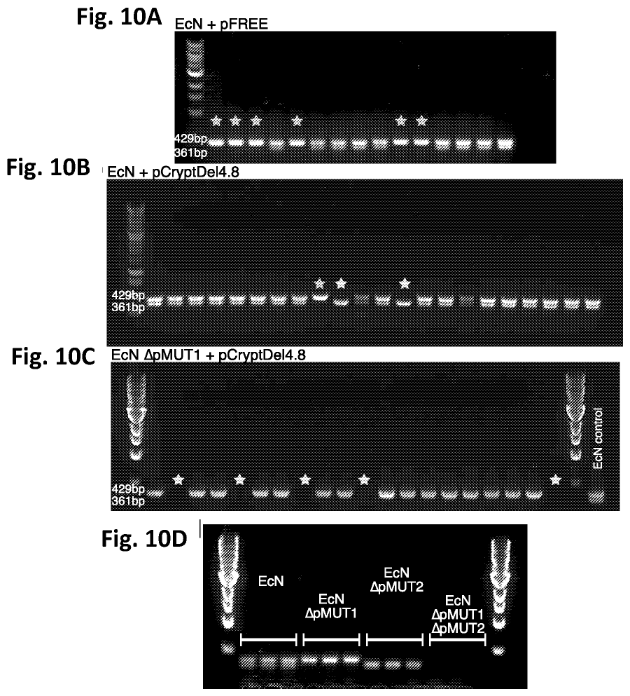

Three gRNA pairs designed and tested to cure pMUT2, however, all designs failed until the antitoxin gene relE found on pMUT2 was included onto the plasmids (FIG. 2D). At each iteration of the design, many pCryptDel variants were tested for their ability to remove pMUT2, and one was identified, labelled pCryptDe14.8, that cured EcN of pMUT2. Upon sequencing, it was found that this plasmid targeted two sites on pMUT2 for Cas9-mediated cleavage, however it also contained an insertion mutation that altered one of the gRNAs targeting the ColE1 origin (FIG. 9). Variants without this mutation could cure pMUT1, but did not cure pMUT2 in any of the colonies tested. pCryptDe14.8 self-cured in all colonies tested, and cured pMUT2 with an efficiency of around 21% (24 out of 117 colonies tested), however it was very poor at curing pMUT1 (3 out of 97 colonies tested). In all cases of pMUT plasmid curing tested, the pFREE and pCryptDe14.8 plasmids were also self-cured during the process. pFREE and pCryptDe14.8 could therefore be used to rapidly produce pMUT plasmid knockout strains through a single overnight growth step. Furthermore, the pFREE and pCryptDel plasmids could used consecutively to create fully pMUT plasmid free EcN (FIG. 10).

### Example 5: Incorporating Genetic Modules for In Vivo Use: Temperature Sensing

Disclosed herein are methods of making plasmid vectors for bacteria in the gut. As such, there are several experimental challenges to both controlling and assessing synthetic genetic systems within the bacteria. Since the bacteria are in the gut of the host organism, they cannot be readily interrogated, and furthermore, due to the complex environment of the gut, it is unlikely that bacteria behave as they do under laboratory conditions. This sets a severe limitation on genetic induction systems that require exogenous chemical inducers in the gut due to the difficulty of supplying a steady inducer concentration. Inducers are normally provided in a concentrated form in the water for the animal, so the effective concentration in the gut is not clear.

However, inducible systems are desirable to simplify cloning and in vitro propagation of DNA and bacterial strains, especially for genes that encode products that are toxic or stress-inducing to the bacteria. Synthetic genetic circuits typically require the bacteria to express heterologous proteins, and these can impose significant metabolic burdens on their host 29. For constitutive high-expressing constructs, given a non-zero mutation rate, any defective mutants that relieve the metabolic burden will quickly come to dominate cultures due to faster growth. Therefore, for in vitro cloning and propagation it is desirable to use inducible systems to create an ‘off’ state where the synthetic system does not significantly reduce fitness during culture propagation. Additionally, the uninduced state provides a further internal control in experiments that can provide valuable insight into the performance of the genetic system.

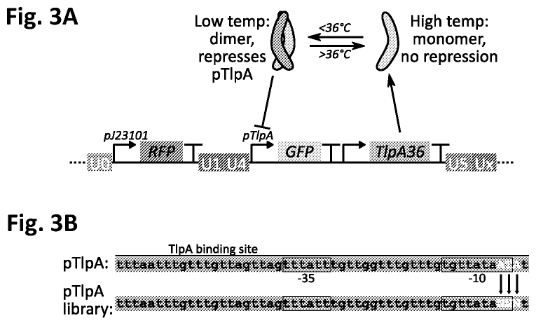

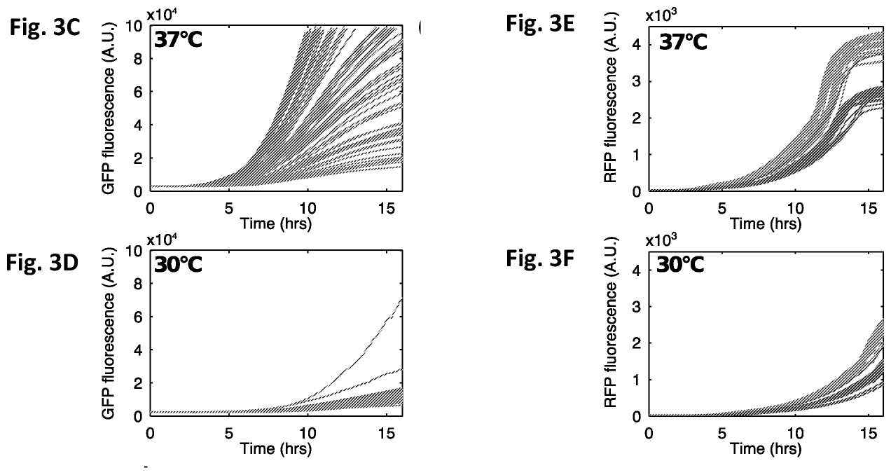

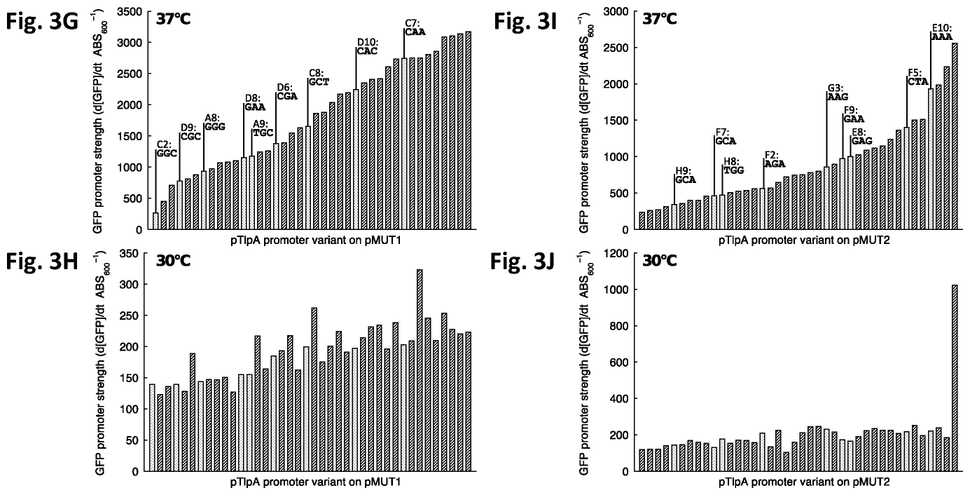

A temperature-sensitive gene expression system from Piraner et al. ³⁰ was implemented, based on the promoter pTlpA and repressor protein TlpA36. TlpA36 forms a dimer at temperatures below 36° C., and this dimer binds pTlpA and prevents gene expression (FIG. 3A). At temperatures above 36° C., the repressor dimer is unstable and does not prevent gene expression. As such, this is an ideal system to provide constitutive high gene expression in vivo, as both human and mouse body temperatures are around 37° C., whilst in vitro the bacteria can be grown at 30° C. To characterize the temperature dependent gene expression, a synthetic ratiometric construct containing pTlpA driving sfGFP was designed, and the constitutive pJ23101 (BioBricks registry) driving mCherry, an RFP (FIG. 3A).

Above a certain critical temperature, the pTlpA promoter is active and acts constitutively, and the promoter was mutated to generate a library with varied expression strengths. The promoter variant could then be selected for a transcriptional unit of interest in order to optimize gene expression. TlpA, from which TlpA36 was derived, binds to the entire pTlpA promoter ³¹ (FIG. 3B), so to minimally disrupt the repressor-promoter interaction mutations were limited to the edge of the promoter region. It was found that the −35 and −10 regions of the promoter are important to RNA polymerase binding and transcription strength using the BPROM software ³². Three mutations in the −10 region of the pTlpA promoter were designed. DNA oligos containing 3 freely varying nucleotides were designed, and used to assemble the ‘sR_TS’ (i.e. “RFP, temperature sensitive”) circuit shown in FIG. 3A on both the pM1s3A and pM2s2A backbones. The assembled plasmids were then transformed into an E. coli cloning strain, Mach1, for an initial screening and characterization. Transformants were initially screened for GFP on LB agar plates grown at 37° C. to select variants with a range of expression strengths, and 40 constructs were chosen each for pM1s3A and pM2s2A. Constructs were labelled by the microwell within the plate, with pM1s3AsR_TS-A1 to D10 for the engineered pMUT1 constructs and pM2s2AsR_TS-E1 to H10 for the pMUT2 constructs. The cells bearing the selected constructs were then monitored during growth in the plate reader, where the GFP, RFP and culture absorbance at both 37° C. and 30° C. was quantified to identify their strength and inducibility (FIG. 3C-3F). It was found that the GFP expression strengths varied significantly between constructs at 37° C., whereas RFP expression remained similar in each case and did not vary as much with temperature.

The promoter strengths of the pTlpA variants (labelled pTlpA-A1 to pTlpA-H10) were found the by calculating the amount of GFP produced per unit time and per cell (FIG. 3G-3J). It was found that the promoters were weaker by around an order of magnitude at 30° C. compared to 37° C., and when induced, covered a wide range of expression strengths on both pMUT vectors. Of these constructs, 9 were selected to cover the range of expression strengths each on the pM1s3A and pM2s2A backbones for sequencing and further use. The selected constructs are highlighted in light green in FIG. 3G-3J, and the mutant nucleotides are shown as labels above. A table with the promoter sequence and their associated expression strengths can be found in Table 7.

TABLE 7
Variant TlpA* promoter sequences, and their promoter strengths,
CsgA-Fe36 production capacity, and CsgA-NbGFP production capacity
at when bacteria were grown on 37° C. and at 30° C. The leftmost
column indicates the host engineered plasmid. Promoter strength
was measured using constructs pM#s#AsR_TS *. Curli material was
produced with plasmids containing the TlpA* variant driving cassette
'csg-Etag' (columns CsgA-FE36), or 'csg-Etag-NbGFP' (columns
CsgA-NbGFP), and curli production was measured using the Congo
Red method (CR) or by immunostaining with anti-Etag on a filter
plate (ELISA).

| | | | | | | | CsgA- | | | | CsgA- | |
|---|---|---|---|---|---|---|---|---|---|---|---|---|
| | Promoter | | Promoter | | CsgA-FE36 | | NbGFP | | CsgA-FE36 | | NbGFP | |
| | variant/ | | strength | | production | | production | | production | | production | |
| | SEQ ID | Promoter | (GFP) | | (CR) | | (CR) | | (ELISA) | | (ELISA) | |
| Plasmid | NO: | sequence | 37ºC | 30° C. | 37º° C. | 30° C. | 37º C. | 30° C. | 37º° C. | 30° C. | 37º C. | 30° C. |
| No plasmid control | | | 65.5 | 58.0 | 1.00 | 1.00 | 1.00 | 1.00 | 0.10 | 0.10 | 0.08 | 0.09 |
| pMUT1 | A8/55 | tttaatttgtttgttagttag | 588.2 | 123.2 | 1.02 | 1.07 | 0.95 | 1.01 | 0.24 | 0.09 | 0.11 | 0.07 |
| | | tttatttgttggtttgtttgt | | | | | | | | | | |
| | | gttataGGGaagc | | | | | | | | | | |
| pMUT1 | A9/56 | tttaatttgtttgttagttag | 721.4 | 137.9 | 1.06 | 1.11 | 1.11 | 1.08 | 0.27 | 0.08 | 0.29 | 0.09 |
| | | tttatttgttggtttgtttgt | | | | | | | | | | |
| | | gttataTGCaagc | | | | | | | | | | |
| pMUT1 | C2/57 | tttaatttgtttgttagttag | 372.7 | 121.3 | 1.00 | 1.07 | 0.95 | 1.00 | 0.11 | 0.08 | 0.07 | 0.08 |
| | | tttatttgttggtttgtttgt | | | | | | | | | | |
| | | gttataGGCaagc | | | | | | | | | | |
| pMUT1 | C7/58 | tttaatttgtttgttagttag | 2402.1 | 179.3 | 1.44 | 1,12 | 0.95 | 1.12 | 0.49 | 0.10 | 0.11 | 0.10 |
| | | tttatttgttggtttgtttgt | | | | | | | | | | |
| | | gttataCAAaagc | | | | | | | | | | |
| pMUT1 | C8/59 | tttaatttgtttgttagttag | 1478.6 | 158.1 | 1.03 | 1.08 | 1.27 | 1.08 | 0.09 | 0.08 | 0.51 | 0.10 |
| | | tttatttgttggtttgtttgt | | | | | | | | | | |
| | | gttataGCTaagc | | | | | | | | | | |
| pMUT1 | D6/60 | tttaatttgtttgttagttag | 1355.9 | 152.6 | 1.32 | 1.17 | 0.47 | 0.08 | | | | |
| | | tttatttgttggtttgtttgt | | | | | | | | | | |
| | | gttataCGAaagc | | | | | | | | | | |
| pMUT1 | D8/61 | tttaatttgtttgttagttag | 618.1 | 128.0 | 1.03 | 1.14 | 1.43 | 1.11 | 0.13 | 0.09 | 0.84 | 0.11 |
| | | tttatttgttggtttgtttgt | | | | | | | | | | |
| | | gttataGAAaagc | | | | | | | | | | |
| pMUT2 | E8/62 | tttaatttgtttgttagttag | 963.6 | 172,2 | 1.41 | 1.10 | 1,32 | 1.08 | 0.35 | 0.08 | 0.54 | 0.10 |
| | | tttatttgttggtttgtttgt | | | | | | | | | | |
| | | gttataGAGaagc | | | | | | | | | | |
| pMUT2 | E10/63 | tttaatttgtttgttagttag | 1016.5 | 196.7 | 1.30 | 1.08 | 1.31 | 1.09 | 0.68 | 0.08 | 0.52 | 0.12 |
| | | tttatttgttggtttgtttgt | | | | | | | | | | |
| | | gttataAAAaagc | | | | | | | | | | |
| pMUT2 | F7/64 | tttaatttgtttgttagttag | 718.1 | 64.4 | 1.25 | 1.03 | 1.00 | 1.00 | 0,19 | 0.09 | 0.29 | 0.09 |
| | | tttatttgttggtttgtttgt | | | | | | | | | | |
| | | gttataGCAaagc | | | | | | | | | | |

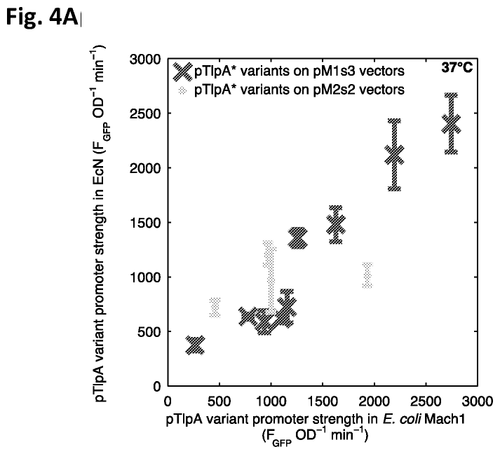

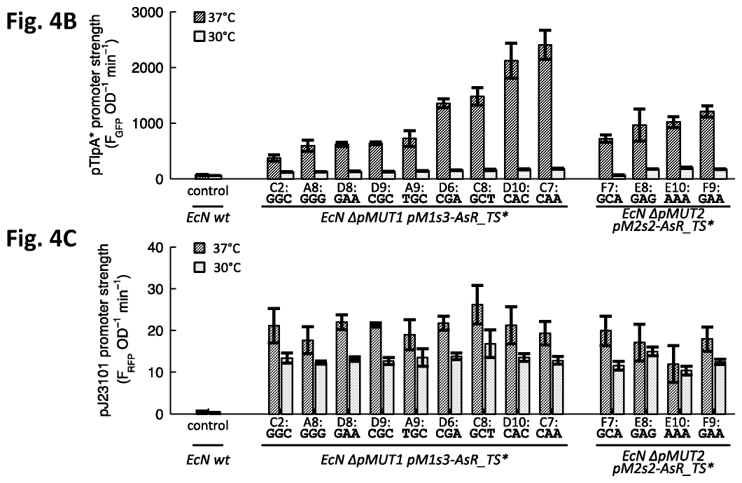

The performance of the pM1s3AsR_TS* and pM2s2AsR_TS* constructs were then characterized in EcN, in each case measuring the engineered pMUT construct performance in the absence of the native cryptic plasmid. It was found that some of the pM2s2AsR_TS* constructs could not be transformed into EcN ΔpMUT2 cells, and thus only 4 of the pMUT2 derived constructs could be characterized. The characterization data from the E. coli Mach1 cloning strain was broadly indicative of performance in EcN (FIG. 4A), although the pM2s2A constructs in particular did not fully match their behaviour in Mach1. The pTlpA* promoters covered a range of expression strengths when induced, and had significantly less expression at 30° C. (FIG. 4B). By contrast, constitutive RFP expression from each construct was similar, and did not vary as much with temperature (FIG. 4C).

### Example 6: Curli Secretion from Engineered pMUT Vectors

Many proteins and peptides have therapeutic potential in the gut³³, and as such the secretion of such peptides into the extracellular space from EcN inhabiting the gut is an attractive approach to therapy. Curli are well-characterized bacterial extracellular matrix proteins, secreted natively by E. coli using dedicated machinery 34 to form robust fibers. Engineered curli systems represent a versatile platform for custom protein materials, as they are capable of tolerating mutations and fusions to functional protein domains, and are consequently being developed as gut therapeutics ³⁵.

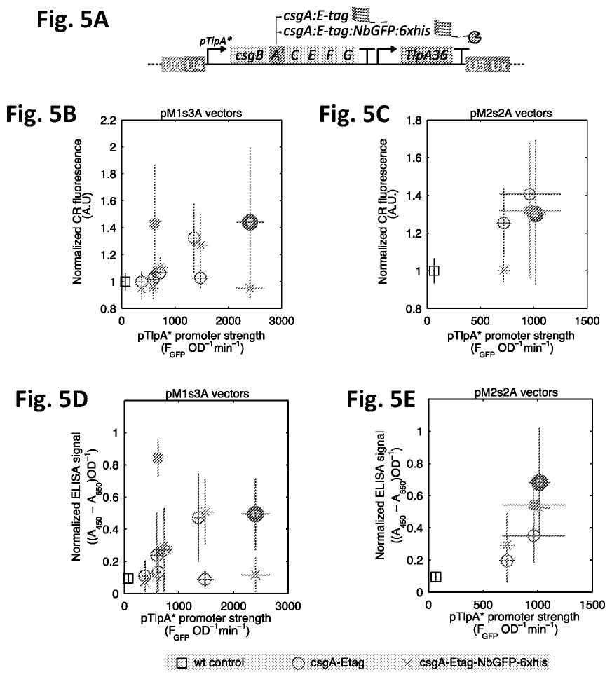

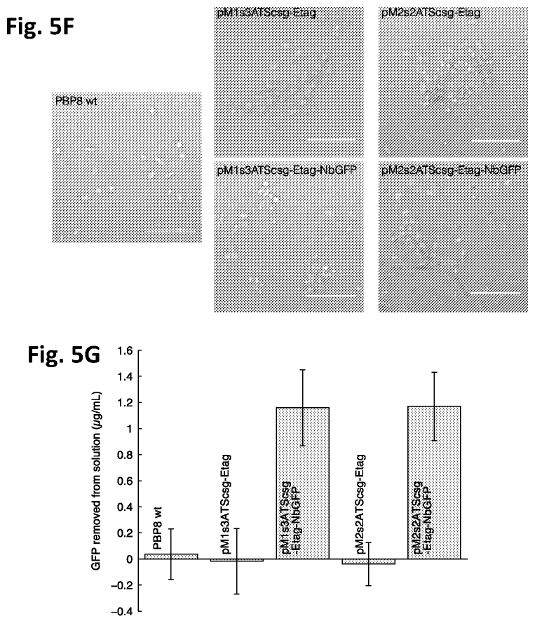

To express curli, a synthetic csgBACEFG operon was used, which encodes the major and minor curli fiber subunits, csgA and csgB, and the secretion machinery necessary for transport from the periplasm to the extracellular space in csgEFG. CsgC prevents intracellular CsgA polymerization, which would be toxic to the bacterium ³⁶. The CsgA monomer was fused to an E-tag epitope tag in a 37 aa flexible linker (FIG. 5A) to enable detection with anti-E-tag antibodies, and this cassette was ‘csg-Etag’. In order to further demonstrate a functional curli variant, constructs where CsgA was fused to a GFP nanobody (NbGFP) ³⁷ were also produced. Nanobodies, also known as VHH domains, are single chain antibody fragments 38, capable of binding tightly to a specific antigen. In this case, the csgA:NbGFP fusion should bind GFP, and due to the insolubility of the curli material, should remove purified GFP from solution. The csgA:NbGFP fusion in cassette ‘csg-Etag-NbGFP’ also encoded an E-tag in a 37 aa flexible linker between the csgA and NbGFP sequences, and the NbGFP was followed by a 6xHis tag.

Overexpression of the csgBACEFG operon can be toxic to cells, and as such the expression strength requires significant tuning to obtain a high yield of curli fibers. Thus, the pTlpA promoter library was used to express a synthetic curli operon to identify an optimal promoter strength. In total, 8 of each ‘csg-Etag’ and ‘csg-Etag-NbGFP’ on pM1s3A vectors, and 3 each on pM2s2A vectors were generated. For each pTlpA*-curli construct, the curli production was characterized using the Congo Red (CR) fluorescence method 39 (FIG. 5B-5C), as well as by a filtration ELISA method with anti-E-tag antibodies (FIG. 5D-5E). Both methods showed similar results, with certain combinations of promoter and csgA variant producing significant yields of curli material. For the csgA:Etag constructs, higher promoter strength generated the higher curli yields, and the highest expressing constructs were chosen to make plasmids pM1s3ATScsg-Etag and pM2s2ATScsg-Etag, which used the pTlpA-C7 and pTlpA-E8 promoters respectively. By contrast, the ‘csg-Etag-NbGFP’ constructs had peak expression at intermediate promoter strengths, with the chosen plasmids pM1s3ATScsg-NbGFP and pM2s2ATScsg-NbGFP containing the pTlpA-D8 and pTlpA-E10 promoters. In all cases, the temperature sensitive curli constructs did not express at 30° C. (Table 7).

At 37° C., the final temperature sensitive curli expression constructs produced curli, which caused the bacterial cultures to aggregate with fluorescent material upon the addition of CR (FIG. 5F). Additionally, at 37° C., the csgA:NbGFP fusions successfully bound and removed purified GFP from solution, demonstrating correct functioning of the nanobody (FIG. 5G).

### Example 7: Engineered pMUTs Performance in the Mouse Gut

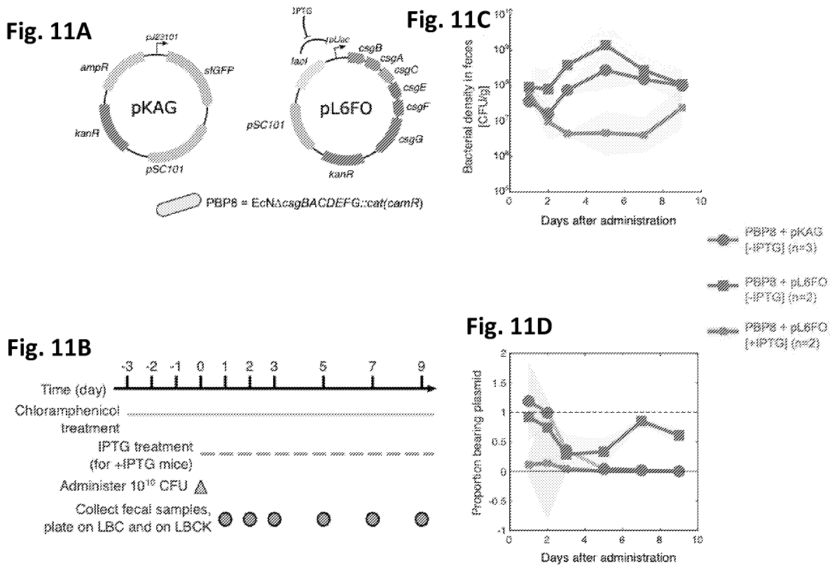

Disclosed herein are methods and compositions to address and improve retention rates of synthetic plasmids in bacteria within the gut. In a preliminary experiment, testing engineered EcN in the mouse gut, it was found that EcN harboring engineered synthetic plasmids were lost during passage through the gut without selection. In the following exemplification, mice were fed PBP8 cells (EcN ΔcsgBACEFG::cat(CamR)) transformed with either plasmid pKAG ⁴⁰, a pSB4K5 based plasmid containing constitutively expressed sfGFP, or pL6FO ³⁹, a similar synthetic plasmid with an IPTG inducible csgBACEFG operon (FIG. 11A). The engineered bacteria were administered to the mice on day 0 of the experiment (FIG. 11B). Both the overall PBP8 population and the plasmid bearing population were tracked in the fecal samples over the subsequent days. In selecting for the PBP8 bacteria by treating with chloramphenicol, it was found that the PBP8 colonized the gut in all cases after administration (FIG. 11C). However, significant plasmid loss for all synthetic plasmids (FIG. 11D) was observed. On day 1 after administration, pL6FO was only present in only around 15% of the population when the curli operon was in the IPTG induced state. Furthermore, for plasmid pKAG, and pL6FO with IPTG induced curli operon, the plasmid bearing bacteria were not found in the gut after 5 days.

Assessment of the plasmid retention of the engineered pMUTs after passing through the mouse gut was investigated; as well as determining the ability of the plasmid system to produce and secrete proteins in an in vivo context, as this feature is key to therapeutic peptide delivery in the gut. Bacterial gene expression in a mammalian gut significantly differs from expression under laboratory conditions 41, and as such in vitro characterization is unlikely to be representative of in vivo functionality.

Typically, it is difficult to assess the gene expression of engineered bacteria in the gut, because they are hard to isolate without in vitro growth that would disrupt any measurement of in vivo gene expression. Direct detection of heterologously-produced proteins in fecal samples is similarly challenging. For most proteins and affinity tags, proteolytic degradation by intestinal proteases is likely to significantly reduce any measurable signal. This is particularly problematic considering the high background signal one can expect from a complex biological medium such as feces. These experimental limitations were, in large part, the motivation to test the pMUT system using curli fibers and VHH domains. In addition to the potential utility of these proteins, both curli and nanobodies are known for their resistance to harsh conditions ^{42,43}, thereby increasing the likelihood of their detection in fecal pellets.

An experiment was designed to test the retention of the engineered pMUTs in vivo, as well as the expression of protein through the plasmid system within the mouse gut. Four plasmids were tested, expressing either cassette ‘csg-Etag’ or ‘csg-Etag-NbGFP’ on pM1s3ATS* or pM2s2TAS* vectors. In each case, PBP8 cells (EcN ΔcsgBACEFG::cat(CamR)) were used, with the native pMUT knocked out whenever the corresponding engineered version was present. As a negative control, PBP8 harboring both wild-type pMUTs with no engineered plasmids were used, making for a total of 5 experimental cohorts. Conditions were labelled as follows:
‘WT pMUTs’ for the control;
‘pM1’ for PBP8 ΔpMUT1 pM1s3ATScsg-Etag;
‘pM1-VHH’ for PBP8 ΔpMUT1 pM1s3ATScsg-Etag-NbGFP;
‘pM2’ for PBP8 ΔpMUT2 pM2s2ATScsg-Etag;
‘pM2-VHH’ for PBP8 ΔpMUT2 pM2s2ATScsg-Etag-NbGFP.

Each of the five bacterial strains were administered to five C57BL/6 mice.

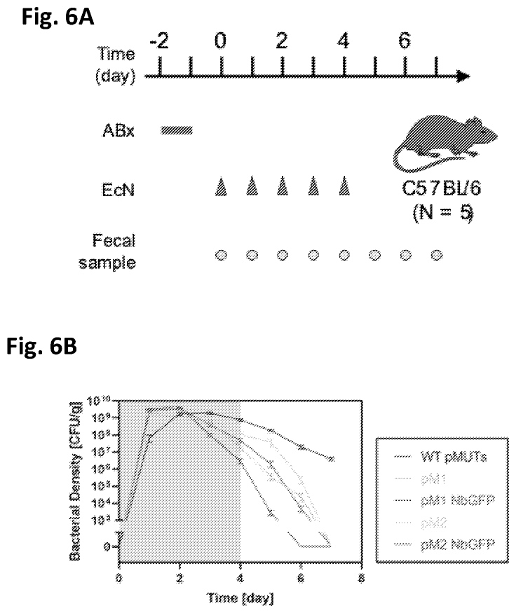

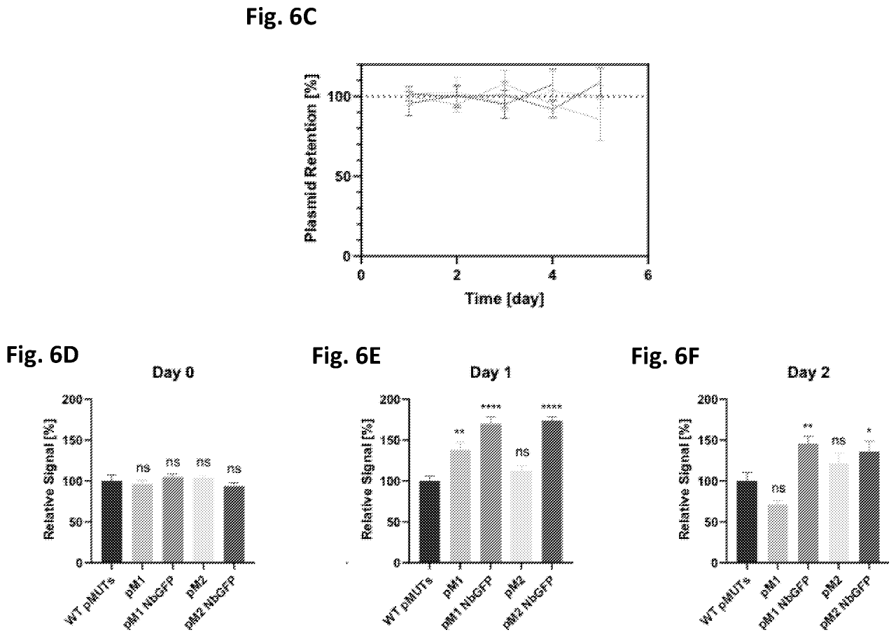

The mice were fed bacterial suspension daily for 5 days and monitored for 3 additional days after cessation of bacterial administration (FIG. 6A). Each day, fecal pellets were collected for colony counting and protein detection. Like most human E. coli isolates, EcN is a poor colonizer of the mouse gut ⁴⁴, though it can transiently colonize mice pre-treated with antibiotics. Therefore, carbenicillin was given two days prior to bacterial feeding, in order to allow the engineered EcN to reach high density, with the antibiotic administration lasting 24 hours to avoid artificially selecting for the engineered plasmids. As a result, EcN density gradually dropped over the course of the experiment, likely due to the recovery of native mouse microbiome (FIG. 6B).

Each fecal pellet was plated on two types of selective plates: chloramphenicol (Cm), selecting for PBP8 irrespective of plasmid presence or identity; and chloramphenicol with carbenicillin (Cm+Carb), which selected specifically for PBP8 with an engineered plasmid. Plasmid retention rates were calculated as the ratio of Cm+Carb to Cm colony counts. All four engineered pMUT cohorts showed no plasmid loss during GI transit, with none of the retention rates differing significantly from 100% (FIG. 6C).

Protein expression was tested via fecal filtration ELISA, modified from a previous protocol 35. In both engineered pMUT1 and pMUT2, significant levels of E-tagged curli fibers were detected (FIG. 6D-6F). Since the mice were fed EcN grown at 30° C., there was no curli expression prior to feeding, so this result demonstrated the ability of the engineered pMUT system to express and secrete proteins in vivo. Interestingly, in both plasmids, the nanobody-containing constructs produced a higher signal than their nanobody-free counterparts. It was suspected that this may be due to the robust CsgA and NbGFP protein domains flanking and protecting the E-tag from proteolysis when in the gut lumen.

### Example 8: Discussion

Provided herein are plasmid vectors based on the E. coli Nissle 1917 pMUT cryptic plasmids that have been characterized for their performance in the mouse gut. Also disclosed herein is the development of a simple method to remove the native pMUT plasmids, and generate reliable pMUT plasmid vectors capable of secreting a functional curli material within the mouse gut without plasmid loss. The pMUT-based plasmid vectors provided herein simplified in vivo experiments by forgoing the need for antibiotics for plasmid maintenance or inducers for gene expression through temperature sensitive circuits.

The pMUT plasmids have no known function, but are stable within EcN during passage through the gut. Notably, and as disclosed herein, the pMUT plasmids can be exploited as vectors for recombinant DNA. Whilst previous studies have used the pMUT plasmids ³, and shown their high plasmid retention in vitro ¹⁹, in vivo efficacy has never been systematically characterized until the present disclosure. In an attempt to cure the native pMUT plasmids, the data of the instant disclosure suggests that pMUT2 stability in EcN is improved by the RelB/RelE toxin/antitoxin system, as pMUT2 could not be cured without expressing the antitoxin gene from the pCryptDel plasmid.

Despite the common use of plasmids in the development of engineered microbes, they are not typically utilized in clinical applications, where exogenous genetic sequences are instead incorporated into the chromosome of the chassis organism. This is mainly due to concerns regarding horizontal gene transfer (HGT), as any antibiotic resistance gene or virulence factor carried on the plasmid would run the risk of being introduced into the host microbiome ⁴⁵. While such concerns are valid for most synthetic plasmids, the unique features of the engineered pMUT system provided herein could address these limitations. Most prominently, the absence of antibiotic selection could eliminate the possibility of spreading resistance genes, as the resistance gene can be excised from any engineered bacterium through a recombinase. Furthermore, the presence of these plasmids in wild-type EcN suggests that the risk posed by any sequence found natively on the plasmid is negligible. Indeed, the safety profile of EcN over decades of probiotic use implies that HGT of pMUT-encoded genes is either exceedingly rare, relatively harmless, or both. Lastly, HGT may be used as a tool for in situ microbiome engineering ⁴⁶. As a selection-free, probiotic-derived plasmid system, the pMUT platform could prove a valuable addition to the toolbox of this emerging microbial intervention strategy. Thus, while the pMUT plasmids could indeed be utilized for the research and development of engineered strains, they could also open the door to plasmid-based production of therapeutics in vivo, in both clinical and preclinical settings.

There are several benefits to using engineered pMUT plasmid vectors compared to genomic incorporation. The first is speed and reliability, since plasmid assembly and transformation are the only steps required for the production of an engineered EcN strain, and this can be done in several days. This can facilitate the rapid construction and development of probiotic bacteria, speeding the development and optimization of prototypes. A further benefit is the ability to incorporate relatively large recombinant genetic constructs with ease. Indeed, one of the largest constructs made was around 13 Kbp (pM2s2ATScsg-NbGFP), incorporating over 7 Kbp of recombinant DNA. Furthermore, both engineered pMUT1 and pMUT2 plasmids could be used simultaneously to house synthetic DNA, allowing for the incorporation of even larger and more complex synthetic DNA systems.

A further benefit to simplifying the process of bacterial engineering is the ability to rapidly and reliably generate multiple variant strains, and thus screen and optimize genetic circuits of interest. The pTlpA promoter library, as exemplified herein, demonstrated how even a relatively small functional change, such as the addition of a fusion protein, can require the redesign of regulatory elements within genetic circuit for optimal function. Notably, the addition of an anti-GFP nanobody required a weaker promoter for curli expression compared to unmodified CsgA-Etag, suggesting that the nanobody reduced secretion efficacy, likely through the toxicity of expression and secretion. However, the weaker expression did not reduce overall curli production in the nanobody constructs, suggesting that curli production was not limited by the expression of the other genes in the csgBACEFG operon.

In the in vivo experiments provided herein, slower clearance of the WT pMUT control strain compared to those expressing proteins through engineered pMUTs was observed (FIG. 6B). This may reflect the added metabolic burden imposed on the cells by overexpression of heterologous protein, rather than any feature of the engineered plasmids, as previous work yielded similar trends with different plasmid systems ³⁵. In addition, bacterial density and protein expression varied between the different conditions, indicating these factors depend on the specific proteins being expressed. Praveschotinunt et al. ³⁵ also demonstrates that PBP8 and WT EcN do not differ substantially in their in vivo behaviour, and that strains expressing wild-type curli fibers can exhibit similar bacterial densities to those producing GFP. These findings suggest that the performance observed by the strains exemplified herein is unlikely to be specific to curli. Taken together, such observations support the compatibility of engineered pMUTs with in vivo expression of a variety of proteins, though the expression strength would have to be adjusted to achieve optimal results for each desired application—as would be the case for any other expression platform, be it genomic integration or plasmid-based.

### REFERENCES

(1) Sonnenborn, U. Escherichia Coli Strain Nissle 1917—from Bench to Bedside and Back: History of a Special Escherichia Coli Strain with Probiotic Properties. FEMS Microbiol. Lett. 2016, 363 (19). https://doi.org/10.1093/femsle/fnw212.
(2) Altenhoefer Artur; Oswald Sibylle; Sonnenborn Ulrich; Enders Corinne; Schulze Juergen; Hacker Joerg; Oelschlaeger Tobias A. The Probiotic Escherichia Coli Strain Nissle 1917 Interferes with Invasion of Human Intestinal Epithelial Cells by Different Enteroinvasive Bacterial Pathogens. FEMS Immunol. Med. Microbiol. 2006, 40 (3), 223-229. https://doi.org/10.1016/S0928-8244(03)00368-7.
(3) Ou, B.; Yang, Y.; Tham, W. L.; Chen, L.; Guo, J.; Zhu, G. Genetic Engineering of Probiotic Escherichia Coli Nissle 1917 for Clinical Application. Appl. Microbiol. Biotechnol. 2016, 100 (20), 8693-8699. https://doi.org/10.1007/s00253-016-7829-5.
(4) Kurtz, C. B.; Millet, Y. A.; Puurunen, M. K.; Perreault, M.; Charbonneau, M. R.; Isabella, V. M.; Kotula, J. W.; Antipov, E.; Dagon, Y.; Denney, W. S.; Wagner, D. A.; West, K. A.; Degar, A. J.; Brennan, A. M.; Miller, P. F. An Engineered E. Coli Nissle Improves Hyperammonemia and Survival in Mice and Shows Dose-Dependent Exposure in Healthy Humans. Sci. Transl. Med. 2019, 11 (475), eaau7975. https://doi.org/10.1126/scitranslmed.aau7975.
(5) Danino, T.; Prindle, A.; Kwong, G. A.; Skalak, M.; Li, H.; Allen, K.; Hasty, J.; Bhatia, S. N. Programmable Probiotics for Detection of Cancer in Urine. Sci. Transl. Med. 2015, 7 (289), 289ra84-289ra84. https://doi.org/10.1126/scitranslmed.aaa3519.
(6) Greenhalgh, K.; Meyer, K. M.; Aagaard, K. M.; Wilmes, P. The Human Gut Microbiome in Health: Establishment and Resilience of Microbiota over a Lifetime. Environ. Microbiol. 2016, 18 (7), 2103-2116. https://doi.org/10.1111/1462-2920.13318.
(7) Riglar, D. T.; Giessen, T. W.; Baym, M.; Kerns, S. J.; Niederhuber, M. J.; Bronson, R. T.; Kotula, J. W.; Gerber, G. K.; Way, J. C.; Silver, P. A. Engineered Bacteria Can Function in the Mammalian Gut Long-Term as Live Diagnostics of Inflammation. Nat. Biotechnol. 2017, 35 (7), 653. https://doi.org/10.1038/nbt.3879.
(8) Whitaker, W. R.; Shepherd, E. S.; Sonnenburg, J. L. Tunable Expression Tools Enable Single-Cell Strain Distinction in the Gut Microbiome. Cell 2017, 169 (3), 538-546.e12.https://doi.org/10.1016/j.cell.2017.03.041.
(9) Riglar, D. T.; Richmond, D. L.; Potvin-Trottier, L.; Verdegaal, A. A.; Naydich, A. D.; Bakshi, S.; Leoncini, E.; Lyon, L. G.; Paulsson, J.; Silver, P. A. Bacterial Variability in the Mammalian Gut Captured by a Single-Cell Synthetic Oscillator. Nat. Commun. 2019, 10 (1), 1-12. https://doi.org/10.1038/s41467-019-12638-z.
(10) Slusarczyk, A. L.; Lin, A.; Weiss, R. Foundations for the Design and Implementation of Synthetic Genetic Circuits. Nat. Rev. Genet. 2012, 13 (6), 406-420. https://doi.org/10.1038/nrg3227.
(11) Theriot, C. M.; Koenigsknecht, M. J.; Carlson, P. E.; Hatton, G. E.; Nelson, A. M.; Li, B.; Huffnagle, G. B.; Z. Li, J.; Young, V. B. Antibiotic-Induced Shifts in the Mouse Gut Microbiome and Metabolome Increase Susceptibility to Clostridium Difficile Infection. Nat. Commun. 2014, 5 (1), 3114. https://doi.org/10.1038/ncomms4114.
(12) Fedorec, A. J. H.; Ozdemir, T.; Doshi, A.; Ho, Y.-K.; Rosa, L.; Rutter, J.; Velazquez, 0.; Pinheiro, V. B.; Danino, T.; Barnes, C. P. Two New Plasmid Post-Segregational Killing Mechanisms for the Implementation of Synthetic Gene Networks in Escherichia Coli. iScience 2019, 14, 323-334. https://doi.org/10.1016/j.isci.2019.03.019.
(13) Kuhlman, T. E.; Cox, E. C. Site-Specific Chromosomal Integration of Large Synthetic Constructs. Nucleic Acids Res. 2010, 38 (6), e92. https://doi.org/10.1093/nar/gkp1193.
(14) Datsenko, K. A.; Wanner, B. L. One-Step Inactivation of Chromosomal Genes in Escherichia Coli K-12 Using PCR Products. Proc. Natl. Acad. Sci. 2000, 97 (12), 6640-6645. https://doi.org/10.1073/pnas.120163297.
(15) Burian, J.; Guller, L.; Mačor, M.; Kay, W. W. Small Cryptic Plasmids of Multiplasmid, Clinical Escherichia Coli. Plasmid 1997, 37 (1), 2-14. https://doi.org/10.1006/plas.1996.1273.
(16) Feldgarden, M.; Golden, S.; Wilson, H.; Riley, M. A. Can Phage Defence Maintain Colicin Plasmids in Escherichia Coli. Microbiology, 1995, 141 (11), 2977-2984. https://doi.org/10.1099/13500872-141-11-2977.
(17) Blum-Oehler, G.; Oswald, S.; Eiteljorge, K.; Sonnenborn, U.; Schulze, J.; Kruis, W.; Hacker, J. Development of Strain-Specific PCR Reactions for the Detection of the Probiotic Escherichia Coli Strain Nissle 1917 in Fecal Samples. Res. Microbiol. 2003, 154 (1), 59-66.
(18) Hacker, J.; Oelschlaeger, T.; Oswald, S.; Sonnenborn, U.; Proppert, H. Plasmid-Free Clone of E. Coli Strain DSM 6601. U.S. Pat. No. 7,993,902B2, Aug. 9, 2011.
(19) Zainuddin, H. S.; Bai, Y.; Mansell, T. J. CRISPR-Based Curing and Analysis of Metabolic Burden of Cryptic Plasmids in Escherichia Coli Nissle 1917. Eng. Life Sci. 2019, 19 (6), 478-485. https://doi.org/10.1002/elsc.201900003.
(20) Hacker, J.; Sonnen-Born, U.; Schulze, J.; Blum-Oehler, G.; Malinka, J.; Proppert, H. Bacterial Plasmids. U.S. Pat. No. 6,391,631B1, May 21, 2002.
(21) Reister, M.; Hof meier, K.; Krezdorn, N.; Rotter, B.; Liang, C.; Rund, S.; Dandekar, T.; Sonnenborn, U.; Oelschlaeger, T. A. Complete Genome Sequence of the Gram-Negative Probiotic Escherichia Coli Strain Nissle 1917. J. Biotechnol. 2014, 187, 106-107. https://doi.org/10.1016/j.jbiotec.2014.07.442.
(22) Tatusova, T.; DiCuccio, M.; Badretdin, A.; Chetvernin, V.; Ciufo, S.; Li, W. Prokaryotic Genome Annotation Pipeline; National Center for Biotechnology Information (US), 2013.
(23) Avison, M. B.; Walsh, T. R.; Bennett, P. M. PUB6060: A Broad-Host-Range, DNA Polymerase-I-Independent ColE2-like Plasmid. Plasmid 2001, 45 (2), 88-100. https://doi.org/10.1006/plas.2000.1511.
(24) Sonnenborn, U.; Schulze, J. The Non-Pathogenic Escherichia Coli Strain Nissle 1917—Features of a Versatile Probiotic. Microb. Ecol. Health Dis. 2009, 21 (3-4), 122-158. https://doi.org/10.3109/08910600903444267.
(25) Gotfredsen, M.; Gerdes, K. The Escherichia Coli RelBE Genes Belong to a New Toxin—Antitoxin Gene Family. Mol. Microbiol. 1998, 29 (4), 1065-1076. https://doi.org/10.1046/j.1365-2958.1998.00993.x.
(26) Hayes, F. Toxins-Antitoxins: Plasmid Maintenance, Programmed Cell Death, and Cell Cycle Arrest. Science 2003, 301 (5639), 1496-1499. https://doi.org/10.1126/science.1088157.
(27) Torella, J. P.; Lienert, F.; Boehm, C. R.; Chen, J.-H.; Way, J. C.; Silver, P. A. Unique Nucleotide Sequence—Guided Assembly of Repetitive DNA Parts for Synthetic Biology Applications. Nat. Protoc. 2014, 9 (9), 2075-2089. https://doi.org/10.1038/nprot.2014.145.
(28) Lauritsen, I.; Porse, A.; Sommer, M. O. A.; Norholm, M. H. H. A Versatile One-Step CRISPR-Cas9 Based Approach to Plasmid-Curing. Microb. Cell Factories 2017, 16. https://doi.org/10.1186/s12934-017-0748-z.
(29) Gyorgy, A.; Jimenez, J. I.; Yazbek, J.; Huang, H.-H.; Chung, H.; Weiss, R.; Del Vecchio, D. Isocost Lines Describe the Cellular Economy of Genetic Circuits. Biophys. J. 2015, 109 (3), 639-646. https://doi.org/10.1016/j.bpj.2015.06.034.
(30) Piraner, D. I.; Abedi, M. H.; Moser, B. A.; Lee-Gosselin, A.; Shapiro, M. G. Tunable Thermal Bioswitches for in Vivo Control of Microbial Therapeutics. Nat. Chem. Biol. 2017, 13 (1), 75-80. https://doi.org/10.1038/nchembio.2233.
(31) Hurme, R.; Berndt, K. D.; Namork, E.; Rhen, M. DNA Binding Exerted by a Bacterial Gene Regulator with an Extensive Coiled-Coil Domain. J. Biol. Chem. 1996, 271 (21), 12626-12631. https://doi.org/10.1074/jbc.271.21.12626.
(32) Salamov, V. S. A.; Solovyevand, A. Automatic Annotation of Microbial Genomes and Metagenomic Sequences. Metagenomics Its Appl. Agric. Nova Sci. Publ. Hauppauge N.Y. USA 2011, 61-78.
(33) Muheem, A.; Shakeel, F.; Jahangir, M. A.; Anwar, M.; Mallick, N.; Jain, G. K.; Warsi, M. H.; Ahmad, F. J. A Review on the Strategies for Oral Delivery of Proteins and Peptides and Their Clinical Perspectives. Saudi Pharm. J. 2016, 24 (4), 413-428. https://doi.org/10.1016/j.jsps.2014.06.004.
(34) Barnhart, M. M.; Chapman, M. R. Curli Biogenesis and Function. Annu. Rev. Microbiol. 2006, 60 (1), 131-147. https://doi.org/10.1146/annurev.micro.60.080805.142106.
(35) Praveschotinunt, P.; Duraj-Thatte, A. M.; Gelfat, I.; Bahl, F.; Chou, D. B.; Joshi, N. S. Engineered E. Coli Nissle 1917 for the Delivery of Matrix-Tethered Therapeutic Domains to the Gut. Nat. Commun. 2019, 10 (1), 1-14. https://doi.org/10.1038/s41467-019-13336-6.
(36) Evans, M. L.; Chorell, E.; Taylor, J. D.; Aden, J.; GOtheson, A.; Li, F.; Koch, M.; Sefer, L.; Matthews, S. J.; Wittung-Stafshede, P.; Almqvist, F.; Chapman, M. R. The Bacterial Curli System Possesses a Potent and Selective Inhibitor of Amyloid Formation. Mol. Cell 2015, 57 (3), 445-455. https://doi.org/10.1016/j.molce1.2014.12.025.
(37) Rothbauer, U.; Zolghadr, K.; Tillib, S.; Nowak, D.; Schermelleh, L.; Gahl, A.; Backmann, N.; Conrath, K.; Muyldermans, S.; Cardoso, M. C.; Leonhardt, H. Targeting and Tracing Antigens in Live Cells with Fluorescent Nanobodies. Nat. Methods 2006, 3 (11), 887-889. https://doi.org/10.1038/nmeth953.
(38) Ghahroudi, M. A.; Desmyter, A.; Wyns, L.; Hamers, R.; Muyldermans, S. Selection and Identification of Single Domain Antibody Fragments from Camel Heavy-Chain Antibodies. FEBS Lett. 1997, 414 (3), 521-526. https://doi.org/10.1016/S0014-5793(97)01062-4.
(39) Kan, A.; Birnbaum, D. P.; Praveschotinunt, P.; Joshi, N. S. Congo Red Fluorescence for Rapid In Situ Characterization of Synthetic Curli Systems. Appl. Environ. Microbiol. 2019, 85 (13), e00434-19. https://doi.org/10.1128/AEM.00434-19.
(40) Rudge, T. J.; Federici, F.; Steiner, P. J.; Kan, A.; Haseloff, J. Cell Shape-Driven Instability Generates Self-Organised, Fractal Patterning of Cell Layers. ACS Synth. Biol. 2013, 2 (12), 705-714. https://doi.org/10.1021/sb400030p.
(41) Heithoff, D. M.; Conner, C. P.; Hanna, P. C.; Julio, S. M.; Hentschel, U.; Mahan, M. J. Bacterial Infection as Assessed by in Vivo Gene Expression. Proc. Natl. Acad. Sci. 1997, 94 (3), 934-939. https://doi.org/10.1073/pnas.94.3.934.
(42) Collinson, S. K.; Emödy, L.; Müller, K. H.; Trust, T. J.; Kay, W. W. Purification and Characterization of Thin, Aggregative Fimbriae from Salmonella Enteritidis. J. Bacteriol. 1991, 173 (15), 4773-4781. https://doi.org/10.1128/jb.173.15.4773-4781.1991.
(43) van der Linden, R. H. J.; Frenken, L. G. J.; de Geus, B.; Harmsen, M. M.; Ruuls, R. C.; Stok, W.; de Ron, L.; Wilson, S.; Davis, P.; Verrips, C. T. Comparison of Physical Chemical Properties of Llama VHH Antibody Fragments and Mouse Monoclonal Antibodies. Biochim. Biophys. Acta BBA—Protein Struct. Mol. Enzymol. 1999, 1431 (1), 37-46. https://doi.org/10.1016/S0167-4838(99)00030-8.
(44) Saito, K. [Studies on the habitation of pathogenic Escherichia coli in the intestinal tract of mice. I. Comparative experiments on the habitation of each type of resistant pathogenic Escherichia coli under an administration of streptomycin]. Paediatr. Jpn. 1961, 65, 385-393.
(45) Lerner, A.; Matthias, T.; Aminov, R. Potential Effects of Horizontal Gene Exchange in the Human Gut. Front. Immunol. 2017, 8. https://doi.org/10.3389/fimmu.2017.01630.
(46) Sheth, R. U.; Cabral, V.; Chen, S. P.; Wang, H. H. Manipulating Bacterial Communities by in Situ Microbiome Engineering. Trends Genet. 2016, 32 (4), 189-200. https://doi.org/10.1016/j.tig.2016.01.005.
(47) Rhodes, J. M. The Role of Escherichia Coli in Inflammatory Bowel Disease. Gut 2007, 56 (5), 610-612. https://doi.org/10.1136/gut.2006.111872.
(48) Mohawk, K. L.; Melton-Celsa, A. R.; Zangari, T.; Carroll, E. E.; O'Brien, A. D. Pathogenesis of Escherichia Coli O157:H7 Strain 86-24 Following Oral Infection of BALB/c Mice with an Intact Commensal Flora. Microb. Pathog. 2010, 48 (3), 131-142. https://doi.org/10.1016/j.micpath.2010.01.003.

1. A method for producing a genetically modified bacterium, comprising introducing into a bacterium at least one engineered cryptic plasmid comprising a heterologous nucleic acid, wherein the heterologous nucleic acid comprises
a nucleic acid sequence encoding a recombinant protein and a polypeptide secretion system for directing the recombinant protein to the outer membrane for secretion,
wherein the bacterium does not comprise any native cryptic plasmids.

2. The method of claim 1, wherein the at least one engineered cryptic plasmid is an engineered pMUT1 or pMUT2.

3. The method of claim 1 or 2, wherein the nucleic acid sequence encoding the recombinant protein and polypeptide secretion system is inserted within a site amplified by a primer pair comprising the sequences set forth in SEQ ID NOs: 21 and 22; SEQ ID NOs: 23 and 24; SEQ ID NOs: 25 and 26; or SEQ ID NOs: 27 and 28.

4. The method of any one of claims 1 to 3, wherein the nucleic acid sequence encoding the recombinant protein and polypeptide secretion system comprises a curli fiber secretion system.

5. The method of any one of claims 1 to 4, wherein the nucleic acid sequence encoding the recombinant protein and polypeptide secretion system comprises a synthetic csgBACEFG operon.

6. The method of any one of claims 1 to 5, wherein the heterologous nucleic acid sequence encodes a recombinant protein fused to a CsgA monomer.

7. The method of any one of claims 1 to 6, wherein the recombinant protein comprises a therapeutic polypeptide selected from the group consisting of an antibody, an antibody fragment, an enzyme, a fusion protein, a hormone, an antigen, a thrombolytic agent, a cytokine, an immunotoxin, and a growth factor.

8. The method of claim 7, wherein the therapeutic polypeptide is an antibody fragment; and the antibody fragment is a single chain antibody, such as a nanobody.

9. The method of claim 8, wherein the single chain antibody is specific for an antigen selected from the group consisting of: carcinogenic embryonic antigen (CEA), glucose transporter 1 (GLUT1), green fluorescent protein (GFP), beta-lactamase, Clostridium difficile Toxin A, Clostridium difficile Toxin B, botulinum toxin (BoTox), cholera toxin (CTX), norovirus capsid protein, rotavirus capsid protein, and Plasmodium membrane protein.

10. The method of claims 7 to 9, wherein the therapeutic polypeptide is fused to an amyloid polypeptide.

11. The method of claim 10, wherein the amyloid polypeptide comprises at least one curli subunit

12. The method of any one of claims 1 to 11, wherein the engineered cryptic plasmid lacks a selectable marker gene.

13. The method of any one of claims 1 to 12, wherein the heterologous nucleic acid is operably linked to an inducible promoter.

14. The method of claim 13, wherein the inducible promoter is responsive to an inducer selected from the group consisting of IPTG, arabinose, tetracycline, and permissive temperature change.

15. The method of claim 13 or 14, wherein the inducible promoter is a temperature sensitive promoter.

16. The method of any one of claims 1 to 15, wherein the bacterium retains the engineered cryptic plasmid in the absence of a selectable marker.

17. The method of any one of claims 1 to 16, further comprising plasmid-curing the bacterium prior to introduction of the engineered cryptic plasmid.

18. An engineered bacterium, comprising at least one engineered cryptic plasmid comprising a heterologous nucleic acid, wherein the heterologous nucleic acid comprises
a nucleic acid sequence encoding a recombinant protein and a polypeptide secretion system for directing the recombinant protein to the outer membrane for secretion,
wherein the bacterium does not comprise any native cryptic plasmids.

19. The engineered bacterium of claim 18, wherein the at least one engineered cryptic plasmid is an engineered pMUT1 or pMUT2.

20. The engineered bacterium of claim 18 or 19, wherein the nucleic acid sequence encoding the recombinant protein and polypeptide secretion system is inserted within a site amplified by a primer pair comprising the sequences set forth in SEQ ID NOs: 21 and 22; SEQ ID NOs: 23 and 24; SEQ ID NOs: 25 and 26; or SEQ ID NOs: 27 and 28.

21. The method of any one of claims 18 to 20, wherein the nucleic acid sequence encoding the recombinant protein and polypeptide secretion system comprises a curli fiber secretion system.

22. The method of any one of claims 18 to 21, wherein the nucleic acid sequence encoding the recombinant protein and polypeptide secretion system comprises a synthetic csgBACEFG operon.

23. The method of any one of claims 18 to 22, wherein the heterologous nucleic acid sequence encodes a recombinant protein fused to a CsgA monomer.

24. The engineered bacterium of any one of claims 18 to 23, wherein the recombinant protein comprises a therapeutic polypeptide selected from the group consisting of an antibody, an antibody fragment, an enzyme, a fusion protein, a hormone, an antigen, a thrombolytic agent, a cytokine, an immunotoxin, and a growth factor.

25. The engineered bacterium of claim 24, wherein the therapeutic polypeptide is an antibody fragment; and the antibody fragment is a single chain antibody, such as a nanobody.

26. The engineered bacterium of claim 25, wherein the single chain antibody is specific for an antigen selected from the group consisting of: carcinogenic embryonic antigen (CEA), glucose transporter 1 (GLUT1), green fluorescent protein (GFP), beta-lactamase, Clostridium difficile Toxin A, Clostridium difficile Toxin B, botulinum toxin (BoTox), cholera toxin (CTX), norovirus capsid protein, rotavirus capsid protein, and Plasmodium membrane protein.

27. The engineered bacterium of any one of claims 24 to 26, wherein the therapeutic polypeptide is fused to an amyloid polypeptide.

28. The engineered bacterium of claim 27, wherein the amyloid polypeptide comprises at least one curli subunit.

29. The engineered bacterium of any one of claims 18 to 28, wherein the engineered cryptic plasmid lacks a selectable marker gene.

30. The engineered bacterium of any one of claims 18 to 29, wherein the heterologous nucleic acid is operably linked to an inducible promoter.

31. The engineered bacterium of claim 30, wherein the inducible promoter is responsive to an inducer selected from the group consisting of IPTG, arabinose, tetracycline, and permissive temperature change.

32. The engineered bacterium of claim 30 or 31, wherein the inducible promoter is a temperature sensitive promoter.

33. The engineered bacterium of any one of claims 18 to 32, wherein the bacterium retains the engineered cryptic plasmid in the absence of a selectable marker.

34. The engineered bacterium of any one of claims 18 to 33, wherein the heterologous nucleic acid further comprises a nucleic acid sequence encoding a polypeptide tag.

35. The engineered bacterium of claim 34, wherein the polypeptide tag is selected from the group consisting of a poly-histidine tag, a myc tag a FLAG tag, a hemagglutinin (HA) tag, and a V5 tag.

36. The engineered bacterium of any one of claims 18 to 35, wherein the engineered bacterium is a non-pathogenic bacterium.

37. The engineered bacterium of any one of claims 18 to 36, wherein the engineered bacterium is a bacterium of the genus Bacteroides or Escherichia.

38. The engineered bacterium of any one of claims 18 to 37, wherein the engineered bacterium is a probiotic bacterium.

39. The engineered bacterium of any one of claims 18 to 38, wherein the engineered bacterium is Escherichia coli.

40. The engineered bacterium of any one of claims 18 to 39, wherein the engineered bacterium is Escherichia coli strain Nissle 1917.

41. The engineered bacterium of any one of claims 18 to 40, wherein the engineered bacterium does not comprise a native csgBACEFG operon.

42. A pharmaceutical composition, comprising the engineered bacterium of any one of claims 18 to 41, and a pharmaceutically acceptable excipient.

43. The pharmaceutical composition of claim 42, wherein the pharmaceutical composition is formulated for oral administration.

44. The pharmaceutical composition of claim 42, wherein the pharmaceutical composition is formulated for rectal administration.

45. The pharmaceutical composition of claim 42, wherein the pharmaceutical composition is formulated as a pill, a capsule, a lozenge, or a suppository.

46. A method of producing a recombinant polypeptide, comprising
culturing the engineered bacterium of any one of claims 18 to 41 under conditions suitable for expression and export of the recombinant polypeptide from the engineered bacterium,
wherein the recombinant polypeptide comprises at least one CsgA subunit and a therapeutic polypeptide.

47. The method of claim 46, wherein expression of the recombinant polypeptide is not toxic to the engineered bacterium.

48. The method of claim 46 or 47, wherein the level of expression and export of the recombinant polypeptide is maintained, as compared to the level of expression and export of the recombinant polypeptide from an engineered bacterium under the same conditions expressed from a conventional plasmid comprising the heterologous nucleic acid sequence and a selectable marker gene.

49. The method of claim of any one of claims 46 to 48, further comprising collecting the recombinant polypeptide from cell culture medium comprising the engineered bacterium.

50. The method of claim 49, wherein the engineered bacterium is not exposed to a lysing agent prior to collecting the recombinant protein from the cell culture medium.

51. The method of claim 49 or 50, wherein the recombinant polypeptide is collected from a supernatant of the cell culture medium.

52. The method of any one of claims 46 to 51, further comprising purifying the recombinant polypeptide.

53. A recombinant polypeptide produced using the methods of any one of claims 46 to 52.

54. A biofilm comprising the recombinant polypeptide produced using the methods of any one of claims 46 to 52.

55. A method for treating a disease or disorder, comprising administering to a subject in need thereof an effective amount of the engineered bacterium of any one of claims 18 to 41 or the pharmaceutical composition of any one of claims 42 to 45,
wherein the engineered bacterium expresses and exports a recombinant polypeptide comprising the at least one CsgA subunit and the therapeutic polypeptide, thereby treating the disease or disorder.

56. The method of claim 55, wherein the engineered bacterium or the pharmaceutical composition is administered orally.

57. The method of claim 55, wherein the engineered bacterium or the pharmaceutical composition is administered rectally.

58. The method of any one of claims 55 to 57, wherein the subject is a mammal.

59. The method of claim 58, wherein the mammal is a human.

60. The method of any one of claims 55 to 59, wherein the disease or disorder is a gastrointestinal disease or disorder.

61. The method of claim 60, wherein the gastrointestinal disease or disorder is selected from the group consisting of inflammatory bowel disease, Crohn's disease, ulcerative colitis, colorectal cancer, ulcer, malabsorption, short-gut syndrome, cul-de-sac syndrome, celiac sprue, tropical sprue, hypogammaglobulinemic sprue, enteritis, short bowel syndrome, and gastrointestinal cancer.

62. The method of any one of claims 55 to 61, wherein the engineered bacterium colonizes the gastrointestinal tract of the subject.

63. The method of claim 62, wherein the engineered bacterium retains the engineered cryptic plasmid for at least 1 to 5 days following administration.

64. A vector, comprising a cryptic plasmid backbone and a heterologous nucleic acid, wherein the heterologous nucleic acid comprises a nucleic acid sequence encoding csgBACEFG operon, and a nucleic acid sequence encoding a therapeutic polypeptide.

65. The vector of claim 64, wherein the csgBACEFG operon is derived from E. coli.

66. The vector of claim 64 or 65, wherein the heterologous nucleic acid is inserted within a site amplified by a primer pair comprising the sequences set forth in SEQ ID NOs: 21 and 22; SEQ ID NOs: 23 and 24; SEQ ID NOs: 25 and 26; or SEQ ID NOs: 27 and 28.

67. The vector of any one of claims 64 to 66, wherein the therapeutic polypeptide is selected from the group consisting of an antibody, an antibody fragment, an enzyme, a fusion protein, a hormone, an antigen, a thrombolytic agent, a cytokine, an immunotoxin, and a growth factor.

68. The vector of claim 67, wherein the therapeutic polypeptide is an antibody fragment, and the antibody fragment is a single chain antibody, such as a nanobody.

69. The vector claim 68, wherein the single chain antibody is specific for an antigen selected from the group consisting of: carcinogenic embryonic antigen (CEA), glucose transporter 1 (GLUT1), green fluorescent protein (GFP), beta-lactamase, Clostridium difficile Toxin A, Clostridium difficile Toxin B, botulinum toxin (BoTox), cholera toxin (CTX), norovirus capsid protein, rotavirus capsid protein, and Plasmodium membrane protein.

70. The vector of any one of claims 64 to 69, wherein the therapeutic polypeptide is fused to an amyloid polypeptide.

71. The vector of claim 70, wherein the amyloid polypeptide comprises at least one curli subunit.

72. The vector of any one of claims 64 to 71, wherein the heterologous nucleic acid further comprises a nucleic acid sequence encoding a polypeptide tag.

73. The vector of claim 72, wherein the polypeptide tag is selected from the group consisting of a poly-histidine tag, a myc tag a FLAG tag, a hemagglutinin (HA) tag, and a V5 tag.

74. The vector of any one of claims 64 to 73, wherein the heterologous nucleic acid is operably linked to an inducible promoter.

75. The vector of claim 74, wherein the inducible promoter is responsive to an inducer selected from the group consisting of IPTG, arabinose, tetracycline, and permissive temperature change.

76. The method of claim 74 or 75, wherein the inducible promoter is a temperature sensitive promoter.

77. The vector of any one of claims 64 to 76, wherein the vector backbone is pMUT1 or pMUT2.

78. The vector of any one of claims 64 to 77, further comprising a nucleic acid encoding a detectable protein.

79. The vector of claim 78, wherein the detectable protein is a fluorescent protein.
